(19) United States
(12) Patent Application Publication
(10) Pub. No.: US 2022/0151952 A1
(43) Pub. Date: May 19, 2022

(54) Novel Nano-Formulation of Cannabidiol (CBD) and Other Cannabinoids for Treatment of Skin Diseases

(71) Applicant: Michael Milane, Orange, CA (US)

(72) Inventor: Michael Milane, Orange, CA (US)

(21) Appl. No.: 17/438,673

(22) PCT Filed: Mar. 13, 2020
(86) PCT No.: PCT/US2020/022755

§ 371 Date: Sep. 13, 2021

Related U.S. Application Data
(60) Provisional application No. 62/817,860, filed on Mar. 13, 2019.

(51) Int. Cl. A61K 31/05; A61K 47/12; A61K 9/16; A61K 9/00; A61K 9/06

(52) U.S. Cl. CPC A61K 31/05; A61K 47/12; A61K 9/06; A61K 9/0014; A61K 9/1617

(57) ABSTRACT

Compositions and methods are disclosed directed to nanoformulations of cannabidiol.

### CROSS-REFERENCE TO RELATED APPLICATION

This application claims priority to U.S. Provisional Application 62/817,860, filed Mar. 13, 2019, which is incorporated herein by reference in its entirety.

### FIELD OF THE INVENTION

Disclosed herein are nano-formulations of cannabidiol (CBD) and other cannabinoids as well as methods of treating specific skin diseases, disorders or conditions using the same. Also disclosed are methods of making such nanoformulations of cannabidiol.

### BACKGROUND OF THE INVENTION

Cannabidiol (CBD) is the major non-psychotropic constituent naturally present in Cannabis sativa L. plant isolated across the 1930s and 1940s, but chemically identified only in the 1960s. (Mechoulam R, et al., Science. 1970; 169:611-2). As well documented from Cannabis sativa L. it is also possible to extract over 100 different cannabinoids compounds considered as its most important bioactive constituents and mainly known for their psychoactive effects. (Elsohly M A, et al., Life Sci. 2005; 78:539-48). Among these the main studied is the 49-tetrahydrocannabinol (Δ9-THC). This class of compounds have their effect mainly by interacting with specific receptors: the cannabinoid receptor type 1 (CB1), found on neurons and glial cells in various parts of the brain, and the cannabinoid receptor type 2 (CB2), found mainly in the body's immune system. (Munro S, et al., Nature. 1993; 365:61-5 Van Sickle M D, et al. Science. 2005; 310:329-32). On the contrary, CBD has a very low affinity for these receptors (100 fold less than Δ9-THC) and when it binds it produces little to no psychoactive effects. (Thomas A, et. al., Br J Pharmacol. 2007; 150:613-23).

CBD is able to exert multiple pharmacological actions via CB1 and CB2 receptors involving intracellular pathways that play a key role in neuronal physiology. (Pertwee R G, et al., International Union of Basic and Clinical Pharmacology. LXXIX. Cannabinoid receptors and their ligands: beyond CB(1) and CB(2). Pharmacol Rev. 2010; 62:588-631; Zuardi A W, et al., Rev Bras Psiquiatr. 2008; 30:271-80). In particular, many actions of CBD seem to be mediated by binding transient receptor potential vanilloid type 1 (TRPV1) (Costa B, et al., Br J Pharmacol. 2004; 143:247-50), G protein-coupled receptor 55 (GPR55) (Pertwee R G, et al., supra; Pertwee R G, Br J Pharmacol. 2007; 152:984-) and 5-hydroxytryptamine receptor subtype 1A (5-HT1A) (Russo E B, et al., Neurochem Res. 2005; 30:1037-43). These additional and novel cannabinoid receptors (CB1 and CB2) have been identified in CB1 and CB2-knockout mice and are expressed in both central and peripheral nervous system. (Buckley N E. Br J Pharmacol. 2008; 153:309-18; Valverde O, et al., Analysis of the endocannabinoid system by using CB1 cannabinoid receptor knockout mice. Handb Exp Pharmacol. 2005; 117-45).

Moreover, CBD and few of the specific cannabinoids have proved to have several anti-inflammatory activities and regulates cell cycle and immune cells functions. (Rieder S A, et al., Immunobiology. 2010; 215:598-605). CBD is able to suppress the production of a wide range of pro-inflammatory cytokines, such as tumor necrosis factor (TNF)-α and interleukin-1 beta (IL-1β), chemokines, growth factors, as well as inhibition of immune cell proliferation, activation, maturation, migration and antigen presentation. (Jean-Gilles L, et al., Immunobiology. 2010; 215:606-1; Mechoulam R, et al., Chem Biodivers. 2007; 4:1678-92). CBD shows also a potent action in inhibiting oxidative and nitrosative stress, modulating the expression of inducible nitric oxide synthase (iNOS) and nitro-tyrosine as well as reducing production of reactive oxygen species (ROS)(Iuvone T, et al., CNS Neurosci Ther. 2009; 15:65-75).

For skin conditions in particular, preclinical and a few clinical trials are showing the efficacy of cannabinoids for various skin conditions (Freidman A, Dermatologist. 02-2019; Milando R, Am J Clin Dermatol. (December 2018); Biro T, Trends Pharmacol Sci. 2009; 30(8):411-420 including atopic dermatitis, psoriasis, acne, scleroderma, skin cancers, neutrophil diseases, dermatomyositis, and cutaneous lupus erythematosus (CLE) (Biro, T., supra; Olah A, et al., J Clin Invest. 2014; 124(9):3713-3724; Ali A, et al., Pak J Pharm Sci. 2015; 28(4):1389-1395; Werth V P, et al. A phase 2 study of safety and efficacy of anabasum (JBT-101), a cannabinoid receptor type 2 agonist, in refractory skin-predominant dermatomyositis. Presented at: 2017 American College of Rheumatology Annual Meeting; Nov. 3-8, 2017; San Diego, Calif.). Cannabinoids also have potential for the treatment of hidradenitis suppurativa, lichen simplex chronicus, prurigo nodularis, among other inflammatory conditions.

Two studies found evidence suggesting that cannabinoids could be used to target acne. Oláh et al showed that CBD inhibited sebocytes and inflammation in immune cells. (Oláh A, et al. J Clin Invest. 2014; 124(9):3713-3724). Similarly, Ali et al found that cannabis 3% extract improved acne symptoms among participants. (Ali A, et al. Pak J Pharm Sci. 2015; 28(4):1389-1395). In autoimmune diseases, phase 3 trials are investigating the efficacy of synesthetic AJA at treating refractory skin-predominant dermatomyotisis and cutaneous systemic sclerosis with results showing efficacy for both conditions. (Werth V P, et al. A phase 2 study of safety and efficacy of anabasum (JBT-101), a cannabinoid receptor type 2 agonist, in refractory skin-predominant dermatomyositis. Presented at: 2017 American College of Rheumatology Annual Meeting; Nov. 3-8, 2017; San Diego, Calif.; Spiera R F, et al. A phase 2 study of safety and efficacy of anabasum (JBT-101), a cannabinoid receptor type 2 agonist, in diffuse cutaneous systemic sclerosis. Presented at: 2017 American College of Rheumatology Annual Meeting; Nov. 3-8, 2017; San Diego, Calif.)

Hence, cannabinoids, including cannabidiol (CBD), are of interest in treating a variety of skin diseases, inflammatory disorders or other dermatology conditions. However, the current clinical applications of topical CBD have been limited by sub-optimal formulations, hence decreasing therapeutic penetration and absorption by the skin. Furthermore, the lipophilic properties of CBD and the tendency to clump on the skin, further complicated the penetration of CBD through the stratum corneum (SC), the main barrier to penetration of the skin in its uppermost layer.

As such, there remains a need for a topical formulation of CBD that has improved percutaneous penetration and therefor an ability to be above the therapeutic index with enhanced penetration and bioavailability, improving efficacy and safety for multiple skin conditions.

### SUMMARY OF THE INVENTION

Disclosed herein are nanoformulations of a cannabidiol (CBD), as well as associated methods including methods of treatment and methods of manufacture. Advantageously, the CBD nanoformulations disclosed herein have improved percutaneous absorption relative to topical formulations of CBD currently available, with improved efficacy and safety.

In one embodiment, a nanoformulation of CBD is disclosed, comprising cannabidiol and at least one fractionated oil (e.g., fractionated coconut oil) comprising medium-chain fatty acids having between 6 and 8 carbon items, wherein the at least one fractionated oil does not contain fatty acids having greater than 8 carbon atoms.

In a particular embodiment, the nanoformulation is an oil-in-water nanoemulsion.

In another particular embodiment, the nanoformulation is a water-in-oil nanoemulsion.

In a particular embodiment, the at least one fractionated oil does not contain fatty acids having greater than 6 carbon atoms.

In a particular embodiment, the nanoformulation further comprises one or more additional ingredients selected from the group consisting of anti-oxidants and penetration enhancers.

In one embodiment, the penetration enhancer is diethylene glycol monoethyl ether and more particularly, diethylene glycol monoethyl ether present in an amount of about 15 wt. %.

In another particular embodiment, the nanoformulation of CBD has a bioavailability greater than about 15%.

In another embodiment, a method of treating a subject in need thereof with the nanoformulation of cannabidiol is disclosed, comprising administering to the subject a therapeutically effective amount of the nanoformulation for treating a skin disease, disorder or condition. The disease or disorder may be non-neoplastic or neoplastic.

In a particular embodiment, the skin disorder is an inflammatory skin disorder. In one embodiment, the inflammatory skin disorder is selected from the group consisted of atopic dermatitis, eczema, purities and itch, pain, psoriasis, acne, scleroderma, skin cancers, rosacea and erythema, vitiligo and inflammatory pigment disorders, neutrophil diseases, dermatomyositis, hidradenitis suppurativa, lichen simplex chronicus, prurigo nodularis, cutaneous lupus erythematosus (CLE) and cosmetic uses secondary to inflammation.

In another particular embodiment, the skin condition is associated with pain. The pain may be acute or chronic.

In one embodiment, the administration is topical and more particularly, to a selected area of the subject's skin surface. In certain embodiments, the selected are is at least one portion of the face, arms or other extremities.

In one embodiment, the method results in more than about 30% absorption of the composition and more particularly, more than about 50% absorption and even more particularly, about 80% or more absorption. In certain embodiments, this absorption occurs in less than 1 hour, less than 30 minutes, less than 10 minutes, less than 5 minutes or less than 1 minute.

In certain embodiments, the subject treated according to the method disclosed herein exhibits a clinically discernible and/or clinically reportable reduction in one or more symptoms of the skin disease or disorder.

In a particular embodiment, the subject treated according to the method disclosed herein exhibits a reduction in erythema, scale, lesion count and/or

In another particular embodiment, the subject treated according to the method disclosed herein exhibits an improvement by investigator gross assessment (IGA).

In a further embodiment, a method of manufacturing the nanoformulation of cannabidiol is disclosed, comprising (i) providing the cannabidiol and at least one fractionated oil comprising medium-chain fatty acids having between 6 and 8 carbon items, wherein the at least one fractionated oil does not contain medium-chain fatty acids having greater than 8 carbon atoms; and (ii) processing the cannabidiol and at least one short-chained fractionated oil to provide a nanomaterial.

### BRIEF DESCRIPTION OF THE FIGURES

FIG. 1 depicts a photograph of a subject with atopic dermatitis who was not responsive to traditional steroid treatments.

FIG. 2 depicts a photograph of the subject of FIG. 1 treated with a composition disclosed BID herein where the subject's facial eczema cleared in 5 days. This subject remained clear for a period of 1 month on monotherapy.

FIG. 3 depicts a photograph of the subject of FIG. 3, following cessation of use of the composition disclosed herein, resulting in the lesions returning in the same regional areas of the face.

FIG. 4 depicts a photograph of a subject with eczema dermatitis (mild), cleared of lesions in 2 days following treatment with a composition disclosed herein as monotherapy. The subject was treated by mono therapy and did not report any safety issues during treatment.

FIG. 5 depicts a photograph of a subject with eczema dermatitis (severed), cleared fully after two weeks of treatment with a composition disclosed herein.

FIG. 6 (a) depicts a photograph of a subject with severe case of atopic dermatitis of the extremities and hands (baseline). FIG. 6 B depicts a photograph of the subject of FIG. 6(a) exhibiting quick onset and resolution of lesions from baseline following two weeks of treatment daily BID with the composition disclosed herein (monotherapy).

FIG. 7 depicts photographs of a subject with atopic dermatitis with lesions on the hands at baseline, as well as at 2 days and 5 days following application of the composition disclosed herein for 5 days BID.

FIG. 8 depicts photographs with a subject with pediatric eczema flaring in the axilla region at baseline, and where lesions were significantly reduced with a single application in a short period of 12 hours. This subject, who has a long history of faring up after playing sports.

FIG. 9 depicts photographs of a subject treated for her atypical rosacea that had not responded to current standard treatment FIG. 10 depicts photographs of the subject of FIG. 9, taken 10 days after BID application of the composition disclosed herein to the facial area twice per day, with a reported 90% reduction of symptoms. She reported resolution of symptoms of erythema and itching.

FIG. 11 depicts the subject of FIG. 10, where administration of the composition disclosed was continued on the same regiment for 3 month exhibiting maintenance clearance of rosacea symptoms.

FIG. 12 depicts a subject with seborrheic dermatitis from baseline to clearance after 4 applications with a QD single application regiment.

FIG. 13 depicts a representative calibration plot of formulation stability per the HPLC method carried out in Example 2. Calibration standards of CBD over a concentration range of 25.0-150 μg/mL were prepared. The limit of quantification (LOQ) of the method was determined to be 0.05 μg/mL (n=3 average S/N of 20) and the limit of detection (LOD) was determined to be 0.025 μg/mL (n=3 average S/N of 9).

FIG. 14 (a) depicts a sample chromatogram representative of a 100 μg/mL calibration standard in sample diluent (RT ca. 19.65 mins). FIG. 14 (b) is a zoomed in version of FIG. 14 (a) to focus on the base of the peak.

FIG. 15 (a) is a sample chromatogram representative of LOD standard (0.025 μg/mL) (RT ca. 19.65 min). FIG. 15(b) is a zoomed in version of FIG. 15(b) to focus on the base of the peak.

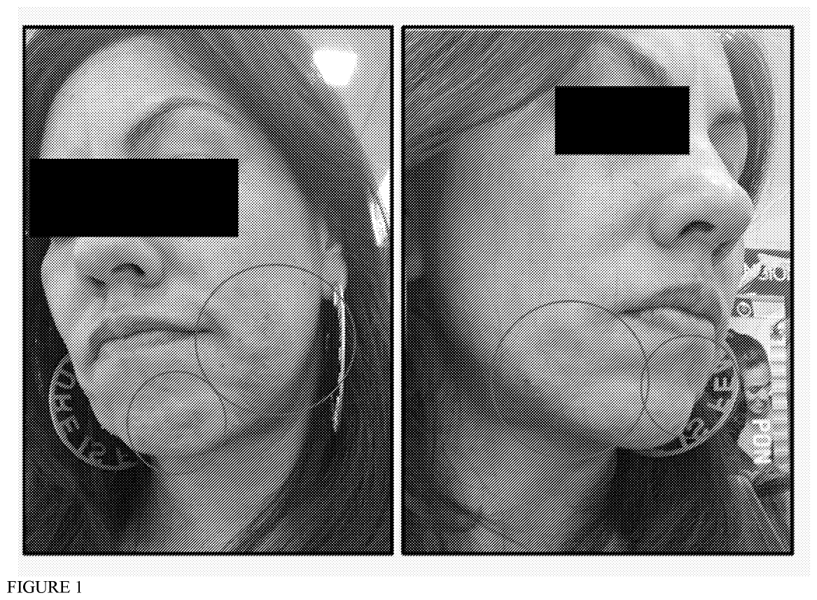

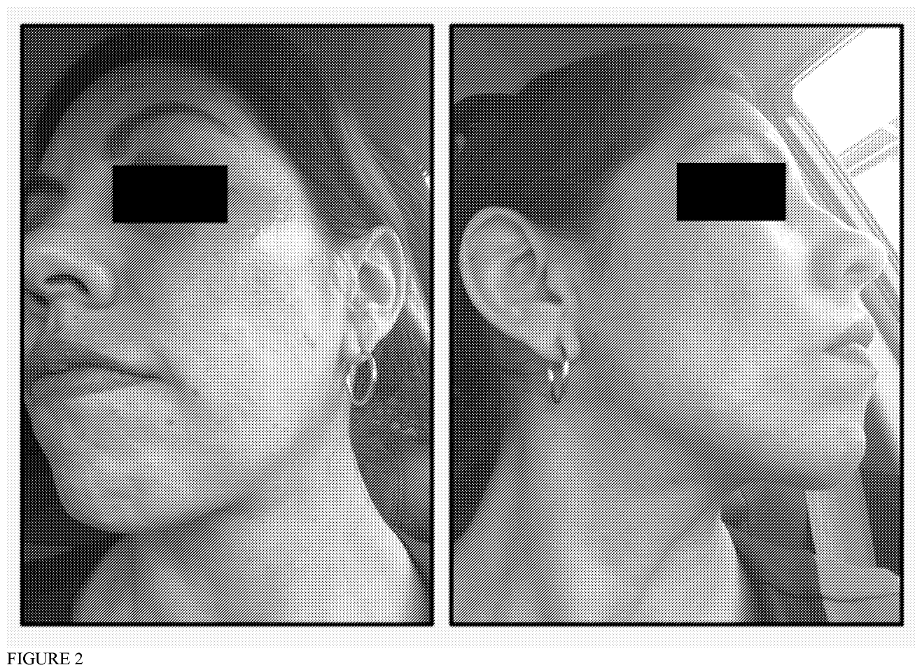

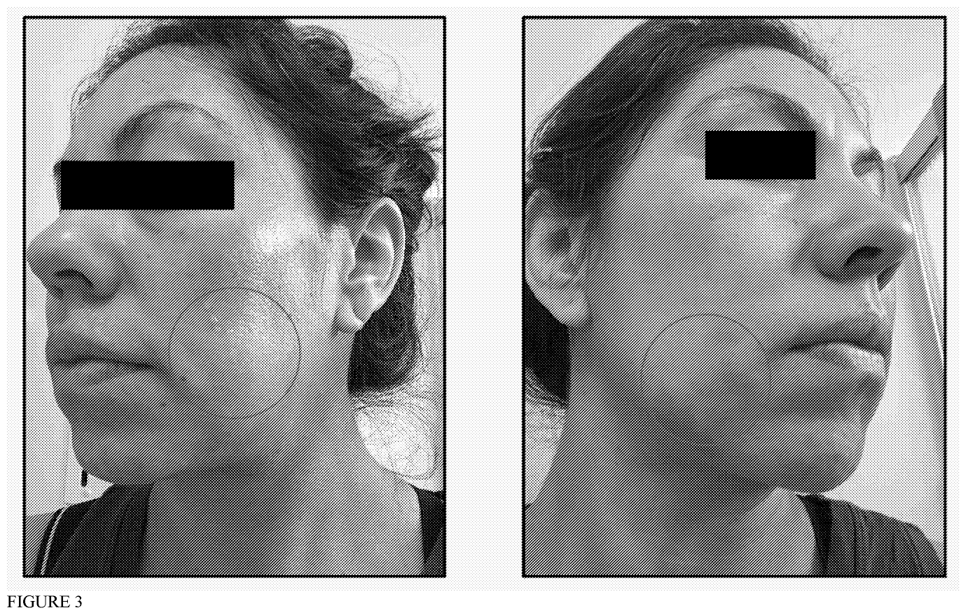

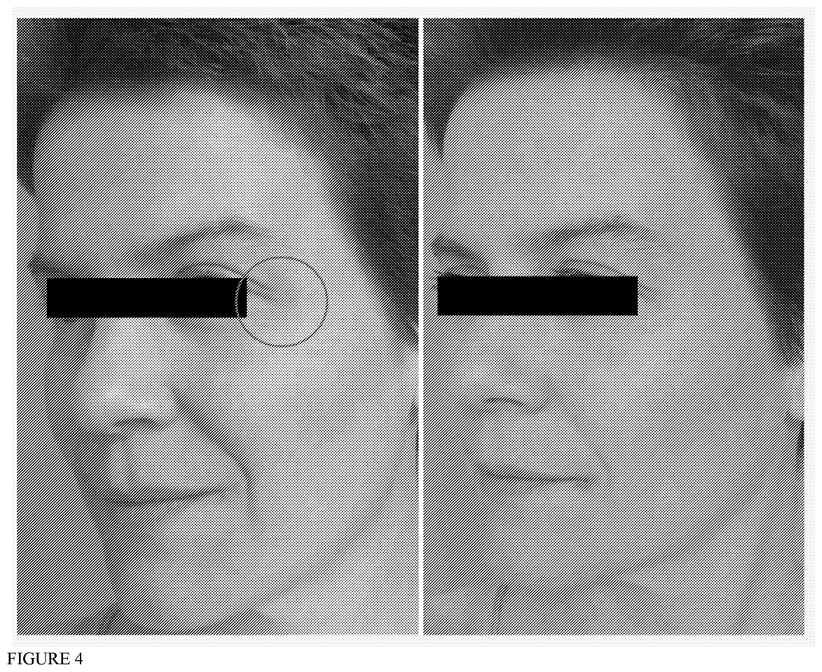

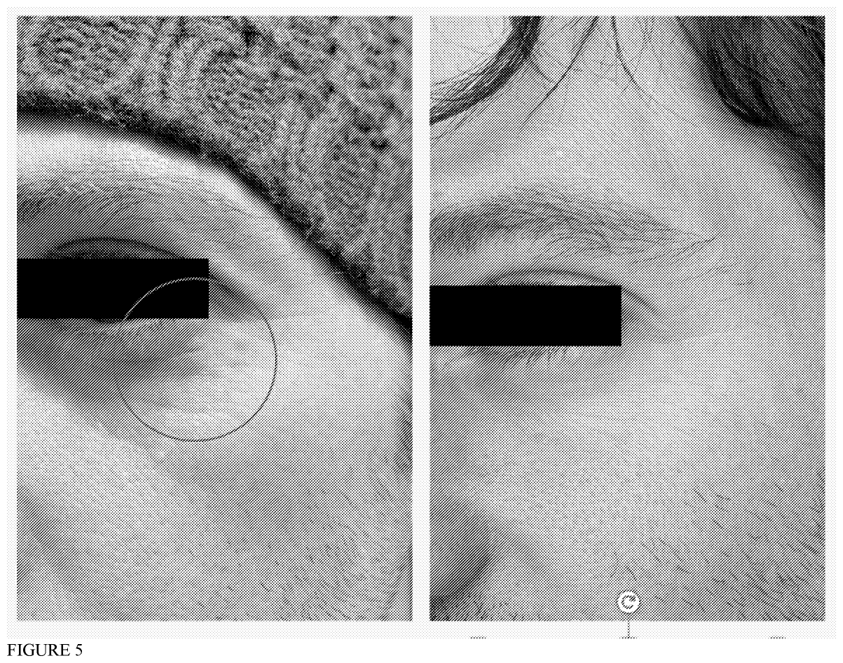

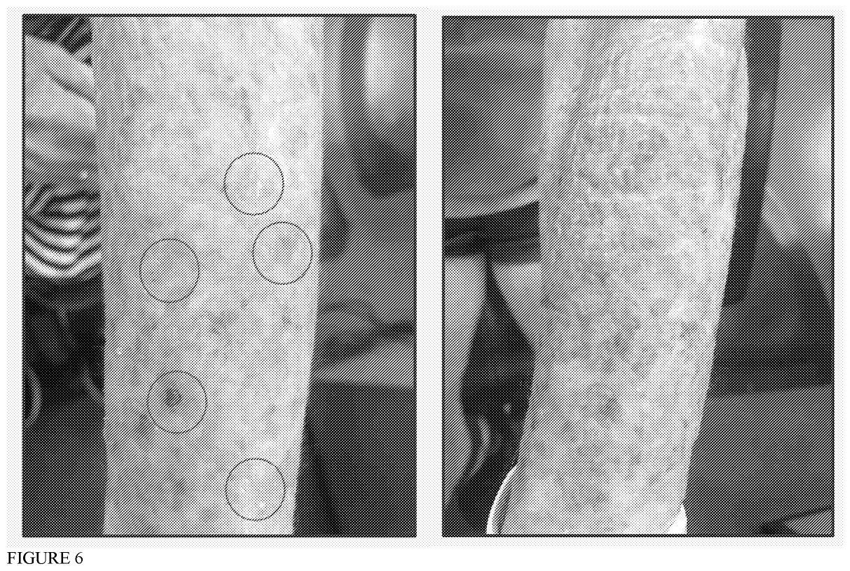

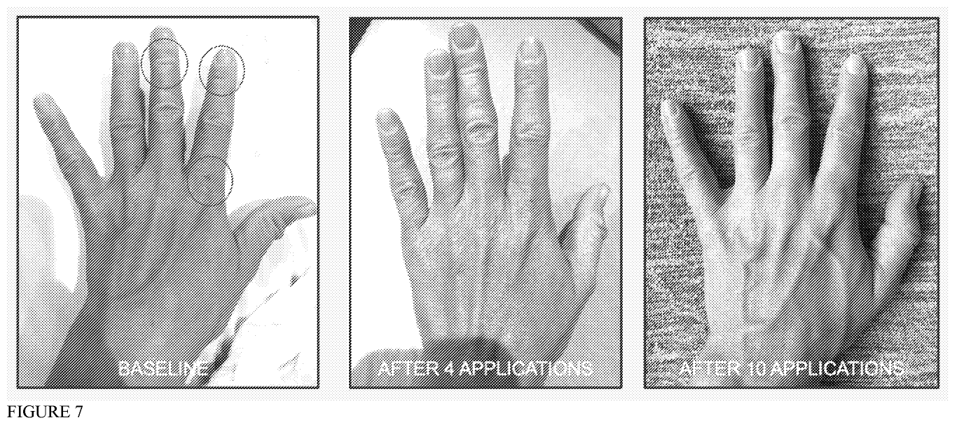

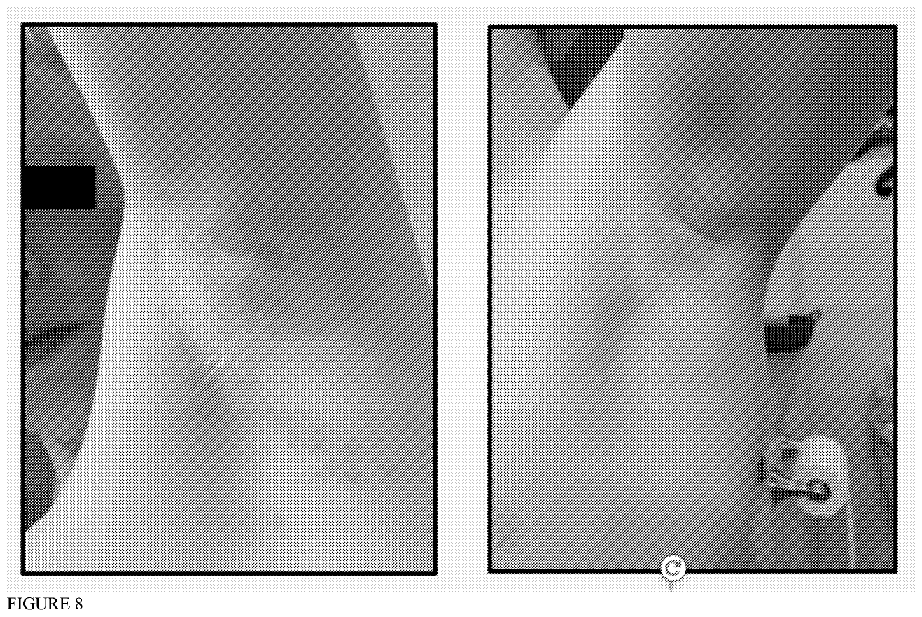

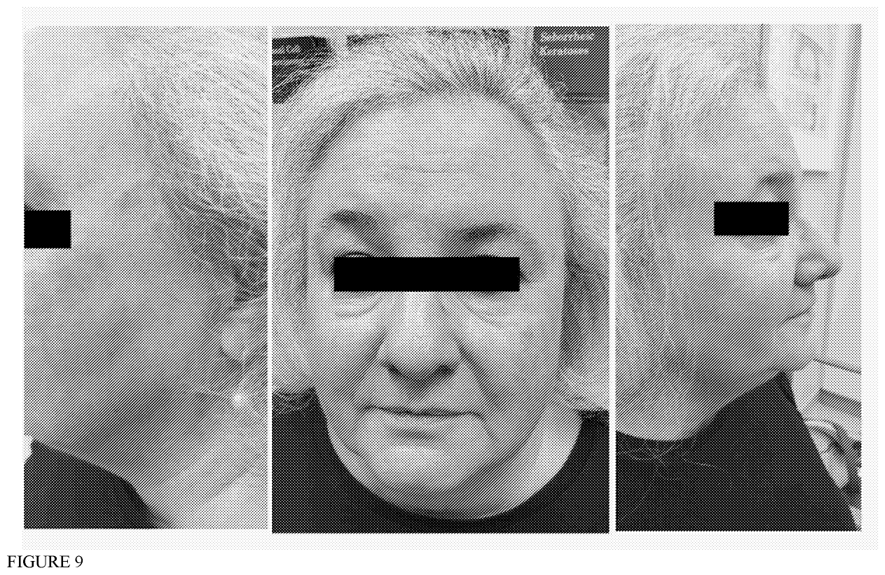

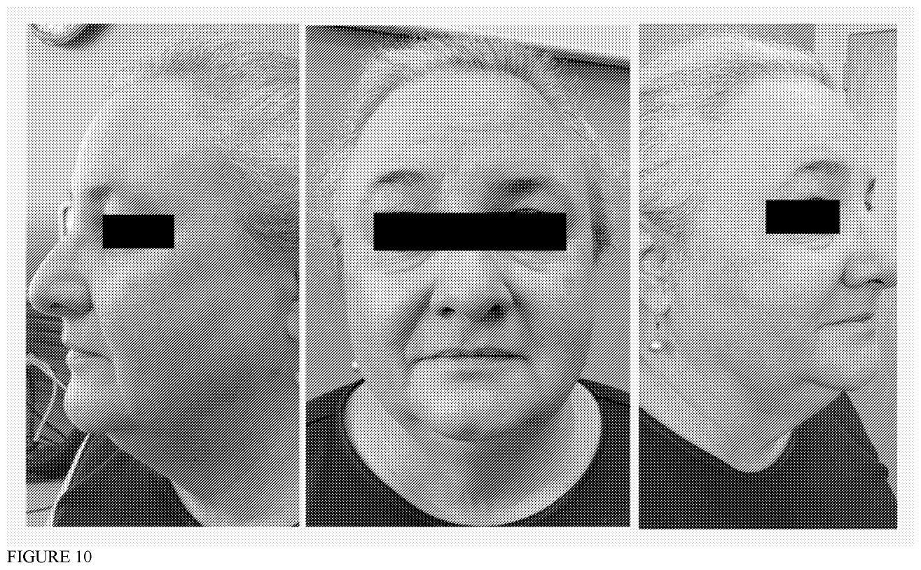

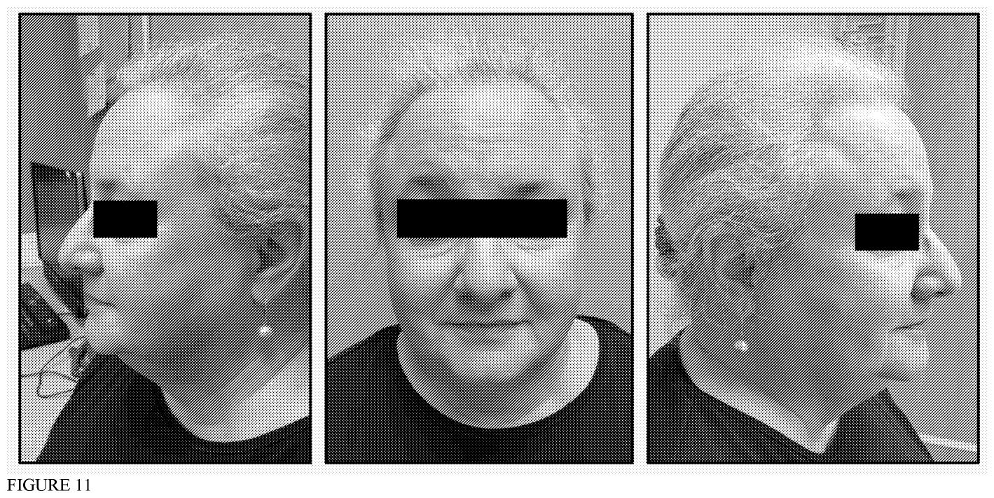

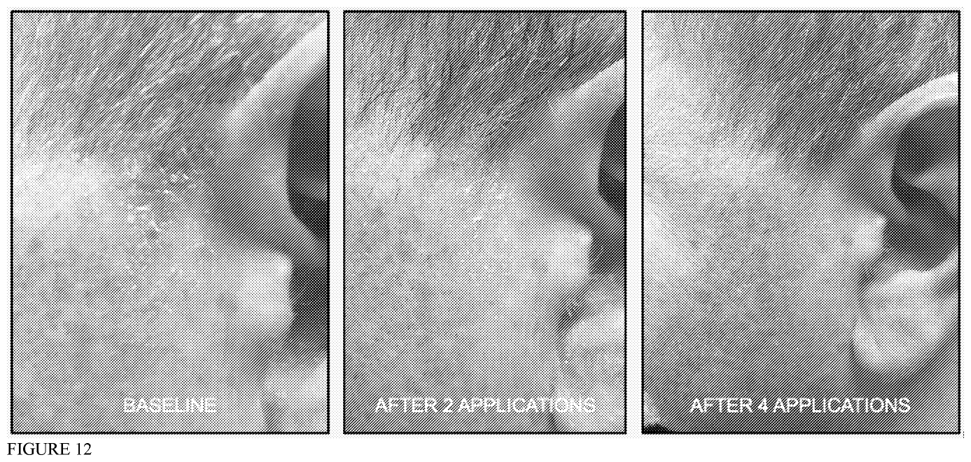

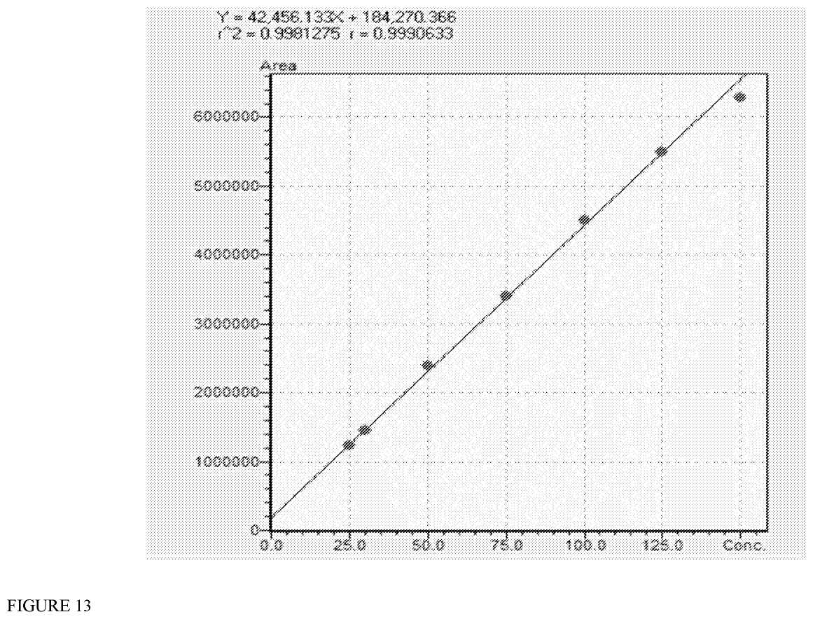

### DETAILED DESCRIPTION OF THE INVENTION

The present invention provides a novel nanoformulation (e.g., nanoemulsion) of Cannabinoids with an optimal barrier delivery formulation, for enhanced therapeutic penetration and bioavailability, as well as methods of use and manufacturing.

In one embodiment, the nanoemulsion disclosed herein is formulation as a cosmetic, dermatological, pharmaceutical or ophthalmological composition.

### I. Definitions

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

The term “acne”, as used herein, refers to all types of acne in all stages, including acne vulgaris observed in adolescents, persistent acne, clinical acne, acne observed in endocrinologic conditions characterized by excess androgen secretion, and the like, in the active inflammatory (pustule-, papule-, comedone-forming) and non-inflammatory (blackhead- and cyst-forming) phases, and post-inflammatory (healing, scarring, and scarred) phase.

The term “amplitude,” as used herein, refers to the maximum variation occurring in an acoustic variable, i.e. how far the variable gets away from its normal, undisturbed value. It is measured in units of pressure: MPa (Mega Pascals)

The term “associated”, as used herein, refers non-covalent interaction between two entities, e.g., molecules, compounds or combinations thereof mediated by one or more of hydrophobic, electrostatic, and van der Walls interactions. The term “bioavailability, as used herein, refers the rate and extent to which a drug reaches at the site of action. The evaluation of topical bioavailability involves quantification of the target tissue itself; that is, one or more components of the skin adjacent to the application site. Non-limiting examples of methods used to measure topical bioavailability include in vitro (human) skin permeation tests, microdialysis (or microperfusion), stratum corneum (SC) tape-stripping, and non-invasive optical/spectroscopic techniques.

The term “BID”, as used herein, refers to administration twice (two times) a day.

The term “cannabis”, as used herein, refers hereinafter to a genus of flowering plants that includes three different species, Cannabis sativa, Cannabis indica and Cannabis ruderalis.

The term “cannabis extract” or “cannabis oil” as used herein refers to a substance obtained by extracting a raw cannabis plant material (e.g., dried hemp, cannabis leaves), using a solvent, wherein the solvent has substantially been removed.

The term “cannabinoid”, as used herein, refers to a large and diverse class of chemical compounds that act on cannabinoid receptors on cells that repress neurotransmitter release in the brain. The most well-studied include tetrahydrocannabinol (THC), cannabidiol (CBD) and cannabinol (CBN). Other known cannabinoids include cannabigerol (CBG) cannabichromene (CBC), cannabicyclol (CBL), cannabivarin (CBV), tetrahydrocannabivarin (THCV); CBDV cannabidivarin (CBDV); cannabichromevarin (CBCV), cannabigerovarin (CBGV); cannabigerol monomethyl ether (CBGM); tetrahydrocannbinolic acid (THCA); cannabidiolic acid (CBDA) and isomers and enantiomers thereof.

The term “carriers”, as used herein, refers to a material suitable for topical drug administration. Carriers and vehicles useful herein include any such materials known in the art, which are nontoxic and do not interact with other components of the composition in a deleterious manner.

The term “cell culture”, as used herein, refers to any in vitro culture of cells. Included within this term are continuous cell lines (i.e., cells with an immortal phenotype), primary cell cultures, finite cell lines (e.g., non-transformed cell lines) and any other cell population maintained in vitro.

The term “clinically discernible”, as used herein, refers to a symptom that can be appreciated by a health care provider.

The term “clinically reportable”, as used herein, refers to a symptom that can be described to a health care provider.

The term “combination therapy,” as used herein, refers to the administration of two or more therapeutic agents to treat a therapeutic condition or disorder described in the present disclosure. Such administration encompasses co-administration of these therapeutic agents in a substantially simultaneous manner or a sequential manner. In either case, the treatment regimen will provide beneficial effects of the drug combination in treating the conditions or disorders described herein.

The term “conjugated”, as used herein, refers to covalent or ionic interaction between two entities, e.g., molecules, compounds or combinations thereof.

The term “cosmetic formulation,” as used herein, refers to formulations intended to alleviate, treat or prevent conditions of the skin, which conditions may, or may not, be considered diseases depending on their severity.

The term “dynamic light scattering” or “DLS” refers to a method used for the measurement of particles and further particle size distribution in an emulsion. Stability studies can also be carried out using DLS. Periodical DLS measurements of a sample can show whether the particles aggregate over time by seeing whether the hydrodynamic radius of the particle increases.

The term “dose”, as used herein, refers to a specified quantity of a pharmaceutical agent provided in a single administration.

The term “dosage unit”, as used herein, refers a form in which a pharmaceutical agent is provided.

The term “effective amount”, as used herein, refers to the administration of an amount of a given compound that achieves the desired effect. An effective amount may be a therapeutically effective amount or a prophylactically effective amount.

The term “emulsifier”, as used herein, refers to surface active molecules and commonly used stabilizers in nanoemulsion preparation to protect small droplets. They reduce the interfacial tension resulting in formation of small and stable nanoemulsions. The emulsifiers also prevent collision and coalescence between the droplets and increases the kinetic stability of the nanoemulsions (Mason et al., 2006). The emulsifiers can be cationic, anionic, nonionic, and zwitterionic in nature. Some examples of emulsifiers are small-molecule surfactants, phospholipids, proteins, and polysaccharides.

The term “emulsion”, as used herein, refers to a dispersion of two immiscible liquids, with the spherical droplets forming the dispersed phase whereas the liquid surrounding it forms the continuous phase. The commonly used liquids to form emulsion are water and oil. The oil droplets dispersed in an aqueous phase are known as oil-in-water (o/w) emulsions. These emulsion systems can be used for the delivery of hydrophobic active substances. The water droplets dispersed in oil are called the water-in-oil (w/o) emulsions and are used for the delivery of hydrophilic compounds. Multiple emulsion systems can also be developed such as the water-in-oil-in-water (w/o/w) and oil-in-water-in-oil (o/w/o) emulsions. Emulsions are categorized as coarse emulsions, microemulsions and nanoemulsions based on their droplet size and stability

The term “energy activation”, as used herein, means activation by an energy source that causes thermal or chemical activity. Energy activation may be by any energy source known in the art. Exemplary energy sources include a laser, ultrasound, acoustic source, flashlamp, ultraviolet light, an electromagnetic source, microwaves, or infrared light. An energy absorbing compound absorbs the energy and become thermally or chemically active.

The term “epidermis”, as used herein, refers to the outermost layer of the skin. It contains five different cell strata. From outside to inside, these are stratum corneum (SC), stratum lucidum, stratum granulosum, stratum spinosum and stratum basale.

The term “fatty acid”, as used herein, refers to a carboxylic acid consisting of a hydrocarbon chain and a terminal carboxyl group, especially any of those occurring as esters in fats and oils. Most naturally occurring fatty acids have an unbranched chain of an even number of carbon atoms, from 4 to 28.

The term “flux,” as used herein, refers to the quantity of the drug permeated into or across skin per unit area per unit time. A typical unit of flux is microgram per square centimeter per hour.

The term “fractionated oil”, as used herein, refers to an oil that has been refined, for example, by removing the long-chain triglycerides, i.e., triglycerides having 14 or more carbons.

The term “highly purified cannabinoids”, as used herein, refers to cannabinoids that have been extracted from the cannabis plant and purified to the extent that other cannabinoids and non-cannabinoid components that are co-extracted with the cannabinoids have been removed, such that the highly purified cannabinoid is greater than or equal to 98% (w/w) pure.

The term “inhibit,” as used herein, refers to prohibiting, preventing, restraining, and lowering, stopping, or reversing progression or severity, and such action on a resultant symptom.

The term “lesion”, as used herein, refers to any single area of altered skin. It may be solitary or multiple. Skin lesions may be flat, elevated above the plane of the skin or depressed below the plane of the skin.

The term “lipid”, as used herein, refers to a group of organic compounds that are esters of fatty acids and are characterized by being insoluble in water but soluble in many organic solvents. Lipids may be classified as: (1) “simple lipids” which include fats and oils as well as waxes; (2) “compound lipids” which include phospholipids and glycolipids; and (3) “derived lipids” such as steroids.

The term “lipid drug conjugate” or “lipoidal prodrug” as used herein refers to a drug covalently bound to a lipid, such as a fatty acid or phospholipid. The bond may be, for example, an ester bond, an amide bond, a disulfide bond or a hydrazine bone. Optionally, a linker or space may be used.

The term “lipid encapsulated”, as used herein, refers to a lipid nanoparticle that provides an active agent or therapeutic agent, with full encapsulation, partial encapsulation, or both.

The term “lipid nanoparticles” or “LPN”, as used herein, refers to lipid-based particles in the submicron range. Lipid nanoparticles can have structural characteristics of liposomes and/or have alternative non-bilayer types of structures. Lipid nanoparticles may comprise one or more lipid species.

The term “liposome”, as used herein, refers to a spherical vesicle of a lamellar phase of the lipid bilayer.

The term “local delivery”, as used herein, refers to tissue specific delivery or distribution.

The term “long-chain fatty acid”, as used herein, refers to a fatty acid containing 14 or more carbon atoms. For example, myristic acid (C14), palmitic acid (C16), stearic acid (C18), arachidic acid (C20) and the like.

The term “medium-chain fatty acid”, as used herein, refers to triglycerides containing fatty acids with between 6 and 12 carbon atoms. For example, caproic acid (C6), caprylic acid (C8), capric acid (C10) and lauric acid (C12).

The term “nanoemulsion”, as used herein, refers to a transparent, monophasic, optically isotropic and kinetically stable colloidal dispersions composed of oil, water, surfactant and cosurfactant with droplet sizes less than 200 nm and in certain embodiments, less than 100 nm. Generally, the small droplet size of nanoemulsion prevents the coalescence of droplets. Nanoemulsion is a translucent systems compared to ordinary emulsions.

The term “nanoparticle,” as used herein, refers to a particle having a diameter, such as an average diameter, from about 10 nm up to but not including about 1 micron, preferably from 100 m to about 1 micron. The particles can have any shape. Nanoparticles having a spherical shape are generally referred to as “nanospheres”.

The term “nanostructured lipid carriers” or “NLC”, as used herein, refers to a colloid system composed of a fluid lipid phase embedded into a solid lipid matrix or localized at the surface of solid platelets and the surfactant layer.

The term “neoplastic,” as used herein, refers to cells having the capacity for autonomous growth, i.e., an abnormal state or condition characterized by rapidly proliferating cell growth. A neoplastic disease state may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The term “onset of action,” as used herein, refers to is the duration of time it takes for a drug's effects to come to prominence upon administration.

The term “penetration enhancer”, as used herein, an agent or a combination of agents that improves the transport of molecules such as a pharmaceutically or cosmetically active agent into or through a natural membrane such as skin or nail.

The term “phytocannabinoids”, as used herein, refers to cannabinoids that originate from nature and can be found in the cannabis plant. The phytocannabinoids can be isolated from plants to produce a highly purified extract or can be reproduced synthetically.

The term “plant oil”, as referred to herein, refers to oils derived from plants as opposed to petroleum or animals. They are triglycerides and contain various fatty acids. Most plant oils are liquid at room temperature.

The term “QD”, as used herein, refers to administration once (one time) per day.

The term “reduction”, “reduce”, “inhibition”, inhibit” or similar terms used herein refers to a clinically reportable and/or clinically discernable decrease for example with respect to one or more symptoms of a disease or disorder. For clarity, this includes both a partial and complete reduction or inhibition. If expressed as an percentage, the decrease may be between 0.1 and about 100%, more particularly, about 0.1%, about 1%, about 5%, about 10%, about 15%, about 20%, about 25%, about 30%, 35%, about 40%, about 45% or about 50% or more.

The term “regular basis”, as used herein, refers to a repeated or repeatable interval of time which can be by way of illustration, a part of a day, daily, twice daily, alternative daily, alternate daily, twice weekly, weekly, fortnightly, monthly or some other repeated or repeatable interval for an appropriate period of time wherein a dose is to be applied. The repeated applications can be determined according to the needs of the subject and the disease or disorder.

The term “skin penetration,” as used herein, refers to the flux of the active ingredient into the different layers of the skin, i.e. the stratum corneum, epidermis and dermis, after application of the nanoformulation to the skin. A solute can diffuse through the skin by three main routes: the trans-appendageal route, the intracellular and intercellular route. Penetration of most topically applied compounds is via the intercellular route.

The term “solid lipid nanoparticle” or “SLN”, as used herein, refers to a nanoparticle composed of lipids that are solid at room temperature with a surface covering of surfactant to stabilize them as a nano-dispersion.

The term “sonication”, as used herein, refers to a subset of mechanical vibration wherein sonic energy generated using a transducer or a probe or other mechanism capable of generating the desired frequency at the desired power, is transmitted to a material. The frequency of such sonic energy may be from 10 KHz to as much as 10 MHz. In this disclosure, when referring to sonication at frequencies less than 20 KHz it is understood that such frequencies are not technically ultrasound as they are in the audible range. As used herein, the term “ultrasonication” refers to sonication using a frequency or frequencies in the inaudible frequency range above about 20 KHz, generally from about 20 KHz to about 1 MHz. As those skilled in the art will appreciate, ultrasonication comprises the transmission of ultrasound energy.

The term “stable” and “stability” are used herein with reference to the shelf-life of a pharmaceutical product, and are related to the physical change, degradation or chemical decomposition of active pharmaceutical ingredients, which limits the shelf-life of a product. Each active pharmaceutical ingredient has its intrinsic stability, its degradation pathways and degradation products, in part depending on the formulation of which it is part, and the storage conditions. The major mechanisms of chemical degradation include oxidation, hydrolysis/dehydration, isomerization/epimerization, decarboxylation, dimerization/polymerization, photolysis and rearrangements. If a product is termed to be “stable” it means in this context that it can be stored for a prescribed time without any of these mechanisms advancing to the extent that compromises product efficacy and safety. A desired nanoemulsion structure, for example, may be characterized by a desired size range, macroscopic observations of emulsion science (is there one or more layers visible, is there visible precipitate), pH, and a stable concentration of one or more the components.

The term “stratum corneum”, as used herein, refers to the thin (20 μm) outermost layer of the skin (epidermis) that primarily mediates the barrier function, which prevents the penetration of all but small (<˜500 Da) and moderately lipophilic molecules. SC consists of keratin-filled corneocytes organized in a matrix of highly ordered multilamellar lipid sheets, described as a brick wall-like structure (the corneocytes forming the bricks and the intercellular lipids representing the mortar. The corneocyte is surrounded by a protein-lipid polymeric envelope. The corneocytes are rigid because of the envelope.

The term “subject”, as used herein, refers to a human or non-human animal selected for treatment or therapy. The age of the subject may vary. In certain embodiments, the subject is an adult, while in others the subject is a pediatric subject. In particular embodiments, the subject has one or more clinically discernible and/or clinically reportable symptoms of a skin disease or disorder.

The term “surfactant”, as used herein, refers any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group. Generally, the amount of surfactant in a nanoemulsion is lower than in a conventional emulsion, e.g., about 5-10% surfactant.

The term “sub-therapeutic dose”, as used herein, refers a dose that is below what is used for treating disease or producing an optimal therapeutic effect.

The term “synthetic cannabinoids”, as used herein, refers compounds that have a cannabinoid or cannabinoid-like structure and are manufactured using chemical means rather than by the plant.

The term “top down”, as used herein, refers to methods of structuring nanomaterial beginning with a bulk solid and obtaining a nanomaterial by structural decomposition. Top down is in contrast to precipitation (bottom up) techniques. Non-limiting examples include high pressure homogenization and milling.

The term “topical administration,” as used herein, refers to administration to skin, orifices, or mucosa. Topical administrations can be administered locally, i.e. they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration. Topical delivery offers several advantages over the conventional dosage forms (oral and intravenous) like avoidance of first-pass metabolism, ease of application, reduces the enzymatic degradation associated with oral delivery, improved patient compliance, and controlled release of drug

The terms “treat”, “treatment” “treating,” or “amelioration”, as used herein, refer to therapeutic treatments, wherein the object is to reverse, alleviate, ameliorate, inhibit, slow down or stop the progression or severity of a condition associated with, a disease or disorder.

The term “therapeutically sufficient flux”, as used herein, refers to permeation flux of the selected drug that delivers sufficient amount of drug into or across the skin to be clinically beneficial. “Clinically beneficial,” when referring to flux, means that at least a portion of the patient population can obtain some degree of benefit from the drug flux.

The term “ultrasound”, as used herein, refers to acoustic radiation with a frequency greater than about 20 kHz, e.g. about 50 kHz, 100 kHz, 500 kHz, 1,000 kHz, 5,000 kHz, 10,000 kHz, or greater. The ultrasound can be medical ultrasound, e.g. about 500 kHz to 30,000 kHz, about 1,000 kHz to 20,000 kHz, about 2,000 kHz to 15,000 kHz, or about 3,000 kHz to 10,000 kHz.

The term “weight percentage”, “weight percent,” “wt. %,” “wt-%,” “percent by weight,” “% by weight,” “percentage”, “percent” or simply “%” and variations thereof, as used herein, refer to the concentration of a substance as the weight of that substance divided by the total weight of the composition and multiplied by 100.

### II. Nanoformulation

Disclosed herein are nanotechnology-based formulations (nanoformulations) of CBD having improved properties relative to CBD formulations known in the art.

In one embodiment, the nanoemulsion formulation comprises a therapeutically effective amount of at least one cannaboid, two immiscible liquids (e.g, water, oil) and an emulsifier. In a particular embodiment, the nanoformulation is a nanoemulsion formulation, wherein the nanoemulsion formulation comprises at least one cannaboid, an aqueous component, an oil component, and a surfactant.

Cannabis extracts are hydrophobic (incompatible with water) and, as such, difficult to deliver to the water-based bloodstream. When consumed orally, for example, they undergo a slow process of gastrointestinal absorption, leading to a delayed onset of action (e.g., from 30 min to 2 hours), as well as a low (10-15%) and unpredictable bioavailability. Currently, most formulation of CBD are limiting in therapeutic efficacy, since most are at a sub-therapeutic dose, secondary to concentration, formulation, purity or delivery method.

The current technology and formulation provide nanoformulations of CBD having improved therapeutic efficacy, while still within the safety standards. In certain embodiments, the nanoformulation significantly enhances the bioavailability of CBD when administered by any route. In particular embodiments, the nanoformulations is characterized by an improved onset of action, and higher levels of therapeutic index. In one embodiment, this activity is increased by a factor 4-6×.

In a particular embodiment, the cannaboid is cannabidiol (CBD).

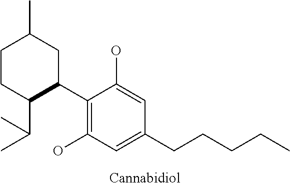

Cannabidiol is one of more than 120 cannabinoids identified in cannabis (marijuana), accounting for up to 40% of the plant's extract. Unlike the main psychoactive cannabinoid in marijuana, tetrahydrocannabinol (THC), CBD does not produce euphoria or intoxication. Cannabidiol has low affinity for the cannabinoid receptors CB1 and CB2 but is believed to act an indirect antagonist thereof. It is generally understood to be safe for human use. CBD is insoluble water but soluble in organic solvents, such as oil.

Cannabidiol from any source is suitable for use.

In a particular embodiment, the nanoformulation contains synthetic or semi-synthetic cannabidiol (e.g., chemically synthesized cannabidiol).

In a particular embodiment, the nanoformulation contains recombination cannabidiol (e.g., cannabidiol produced in yeast or other suitable host).

In a particular embodiment, the nanoformulation contains phytogenic cannabidiol, e.g., cannabidiol derived from plants, such as Cannabis sativa and Cannabis indica.

In one embodiment, the phytogenic cannabidiol is derived from a cannabis cultivar that is CBD dominant, i.e., produces higher levels of CBD (and/or CBDA) than THC (and/or THCA). In another embodiment, the phytogenic cannabidiol derived from a cannabis cultivar that is CBD-rich.

In certain embodiments, the cannabis cultivar is characterized by greater than about 10%, greater than about 15%, greater than about 20%, greater than about 25%, greater than about 30%, greater than about 35%, greater than about 40%, greater than about 45% or greater than about 50% or more CBD.

In other embodiments, the cannabis cultivar is characterized by less than about 1.0%, less than about 0.5% or less than about 0.3% THC.

In certain embodiments, the cannabis cultivar is characterized by a CBD:THC ratio of greater than about 20:1, greater than about 25:1 or greater than about 30:1.

In another particular embodiment, the nanoformulation contains synthetic cannabidiol. In another embodiment, the cannaboid is cannabinol, cannabichromine or cannabigerol.

The amount of cannabidiol present in a nanoformulation of the present invention will be an amount effective to treat a given skin condition based on observational studies or to prevent the same. In certain embodiments, the amount or concentration of cannabidiol is at least about 0.5% to 1% by weight based on the total weight of the nanoformulation.

In a particular embodiment, the amount or concentration of cannabidiol is between about 0.5% and about 20%, about 1% and about 10% or about 2% to about 5% by total weight of the formulation.

In another particular embodiment, the amount or concentration of cannabidiol is about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10% or more by weight based on the total weight of the nanoformulation.

In a particular embodiment, the amount or concentration of cannabidiol is between about of 25.0-150 μg/mL and more particularly, about 50 and about 125 μg/mL, more particularly about 75 and about 100 μg/mL.

In certain embodiments, the amount or concentration is about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 μg/mL or more.

Exemplary embodiments disclosed herein contain CBD, but the disclosed formulation can incorporate any/all cannabinoids, due to similar physical and chemical properties. CBD is lipophilic and stored in the short-chained fractionated coconut oil, therefore the release of the CBD may be improved when the drug is present in the dispersed outer phase of an Nano emulsion. To achieve this, it was considered that the inclusion of water-in-oil formulations should also be assessed (where the oil phase is the dispersed phase and contains the lipophilic Cannabinoid and short-chained fractionated coconut oil mixture). However, since water in oil emulsions are more challenging to formulate as they are very sensitive to changes in manufacturing procedure and are often less physically stable than their oil-in-water counterparts, both oil in water and water in oil formulations were assessed for the best results.

In a particular embodiment, the nanoformulation further comprises at least one additional active agent, i.e., an active agent other than cannabidiol. In one embodiment, the additional active agent is a cannabinoid other than cannabidiol. The additional active agent may be, without limitation, a combination of any of the following cannabinoids:
THC (tetrahydrocannabinol, THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin, CBT (cannabicitran), or others as identified for combination.

### Main Classes of Natural Cannabinoids

| Type | Skeleton | Cyclization |
|---|---|---|
| Cannabigerol-type CBG | 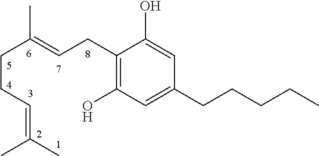 [SMILES: CCCCCc1cc(O)c(C/C=C(\C)CCC=C(C)C)c(O)c1] | 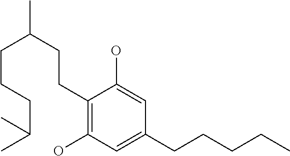 [SMILES: CCCCCc1cc(O)c(CCC(C)CCCC(C)C)c(O)c1] |
| Cannabichromene-type CBC | 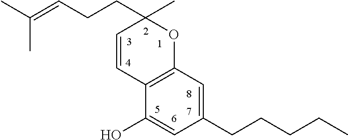 [SMILES: CCCCCc1cc(O)c2c(c1)OC(C)(CCC=C(C)C)C=C2] | 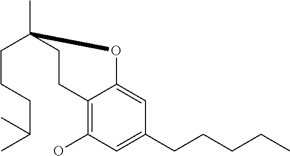 |
| Cannabidiol-type CBD | 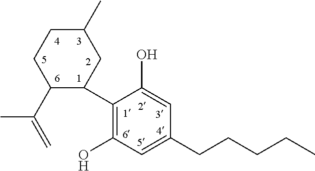 [SMILES: C=C(C)C1CCC(C)CC1c1c(O)cc(CCCCC)cc1O] | 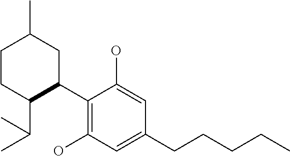 |
| Tetrahydrocannabinol- and Cannabinol-type THC, CBN | 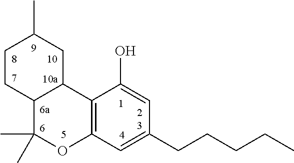 [SMILES: CCCCCc1cc(O)c2c(c1)OC(C)(C)C1CCC(C)CC21] | 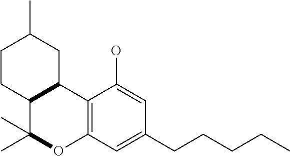 |
| Cannabielsoin-type CBE | 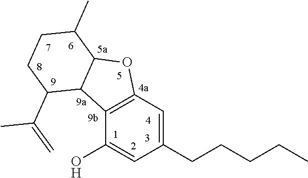 [SMILES: C=C(C)C1CCC(C)C2Oc3cc(CCCCC)cc(O)c3C12] | 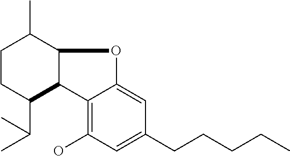 |
| iso- Tetrahydrocannabinol- type iso-THC | 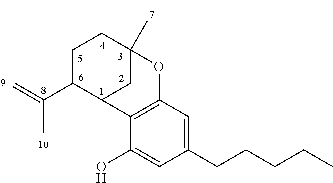 [SMILES: C=C(C)C1CCC2(C)CC1c1c(O)cc(CCCCC)cc1O2] | 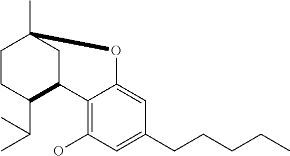 [SMILES: CCCCCc1cc(O)c2c(c1)O[C@]1(C)CC[C@H](C(C)C)C2C1] |
| Cannabicyclol-type CBL | 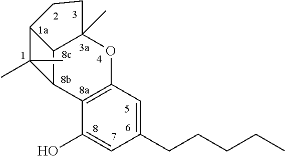 | 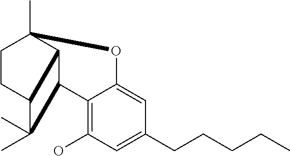 |
| Cannabicitran-type CBT | 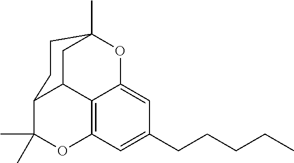 [SMILES: CCCCCc1cc2c3c(c1)OC(C)(C)C1CCC(C)(CC31)O2] | 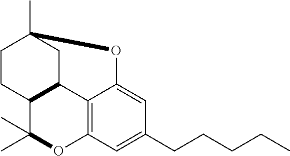 |

In certain embodiments, the therapeutic effect of the cannabidiol and the at least one additional active agent (e.g., the cannabinoid other than cannabidiol) is synergistic.

In one embodiment, the nanoformulation comprises a nanomaterial, such as a nanocarrier or nanoparticle. The size of the nanomaterial may vary. In one embodiment, the nanomaterial is between about 20 and about 200 nm, about 40 nm and about 100 nm, more particularly, about 20 and about 100 nm, about 30 and about 100 nm, 40 and about 100 nm, about 50 nm and about 100 nm, more particularly, about 45 nm and about 55 nm.

In a particular embodiment, the nanomaterial is about 40 nm, about 45 nm, about 50 nm, about 55 nm, about 60 nm, about 65 nm, about 70 nm, about 75 nm, about 80 nm, about 85 nm, about 90 nm, about 95 nm or about 100 nm.

In another particular embodiment, the nanomaterial is about 1 nm, about 5 nm, about 10 nm, about 15 nm, about 20 nm, about 25 nm, about 30 nm, about 35 nm.

Cannabidiol may be associated with the nanomaterial is any suitable manner. In one embodiment, the cannabidiol is adsorbed or covalently attached to the nanocarrier's surface. In another embodiment, the cannabidiol is encapsulated within the nanomaterial, either completely or partially.

The nanocarrier may be, for example, organic (e.g., polymers, lipids), inorganic (e.g., metals, metal oxides) carbon-based.

In a particular embodiment, the nanocarrier is organic and more particularly, a lipid-based nanoparticle (LPN). The term “lipid-based”, as used herein, refers to compositions that primarily comprise lipids.

The lipid-based nanoparticle may vary in size. In a particular embodiment, the lipid-based nanoparticle has a mean particle size of between about 40 nm and about 100 nm, more particularly, about 50 nm and about 100 nm, even more particularly, about 45 nm and about 55 nm. In one embodiment, the lipid-based nanoparticle has a narrow polydispersity index (PI), e.g., lower than 0.2.

In a particular embodiment, the lipid-based nanoparticle is a liposome, micelle, flexible vesicle, lipoplex nanoparticle, lipid-drug conjugate (LDC), lipid nanocapsule (LNC), solid lipid nanoparticle (SLN) or nanostructured lipid carrier (NLC). In one embodiment, the cannabidiol is lipid-encapsulated. Alternatively, the lipid-based nanoparticle is nanoemulsion.

In another particular embodiment, the lipid-based nanoparticle is a nanostructured lipid carrier (NLC). In NLCs, the lipidic phase contains both solid (fat) and liquid (oil) lipids at ambient temperature. Compared to SLNs, NLCs possess lower melting point due to their oil content, while maintaining their particulate character and being solid at body temperature. In one embodiment, the NLC may be a Class I (imperfect type), class II (formless type) or class III (multiple type).

In certain embodiments, the aqueous component of the nanoformulation is selected from distilled water, deionized water, normal saline, phosphate buffered saline and mixtures thereof.

In certain embodiments, the oil phase comprises a fractionated oil.

In certain embodiments, the formulation comprises a cannabidiol and a lipid. The cannabidiol is lipophilic, so will be stored in the lipid.

Any suitable lipid may be used. The lipid may be synthetic, semi-synthetic or a naturally-occurring lipid.

In one embodiment, the lipid is at least one fractionated oil. Fractionated oils containing fatty acids having between 4 and 8 carbon atoms, and more particularly, between 6 and 8 carbon atoms, have been determined to be advantageous to the oils having fatty acids with more than 8 carbon atoms. Fatty acids having between 6 and 8 carbon atoms are generally referred to as medium-chain fatty acids (MCFA). Fatty acids having more than 12 carbon atoms are generally referred to as long-chain fatty acids (LCFA).

Butyric (butanoic acid): CH₃(CH₂)₂COOH is a saturated fatty acid having 4 carbon atoms (C4:0); caproic (hexanoic acid): CH₃(CH₂)₄COOH is a saturated fatty acid having 6 carbon atoms (C6:0); caprylic (octanoic acid): CH₃(CH₂)₆COOH is a saturated fatty acid having 8 carbon atoms (C8:0).

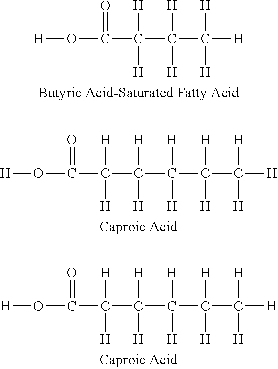 [SMILES: CCCC(=O)O ; CCCCCC(=O)O ; CCCCCC(=O)O]

Capric (decanoic acid): CH₃(CH₂)₈COOH is a saturated fatty acid having 10 carbon atoms (C10:0); auric (dodecanoic acid): CH₃(CH₂)₁₀COOH is a saturated fatty acid having 12 carbon atoms (C12:0); myristic (tetradecanoic acid): CH₃(CH₂)₁₂COOH is a saturated fatty acid having 14 carbon atoms (C14:0); palmitic (hexadecanoic acid): CH₃(CH₂)₁₄COOH is a fatty acid having 16 carbon atoms (C16:0); stearic (octadecanoic acid): CH₃(CH₂)₁₆COOH is a fatty acid having 18 carbon atoms (C18:0); arachidic (eicosanoic acid): CH₃(CH₂)₁₈COOH is a fatty acid having 20 carbon atoms (C20:0); behenic (docosanoic acid): CH₃(CH₂)₂₀COOH or C22:0

In a particular embodiment, the fractionated oil is enriched for fatty acids containing fatty acids having between 4 and 8 carbon atoms, and more particularly, between 4 and 6 carbon atoms or between 6 and 8 carbon atoms.

In a further particular embodiment, the fractionated oil is enriched for fatty acids having 6 carbon atoms.

In a particular embodiment, the fractionated oil contains fatty acids, wherein more than 80%, more than 85%, more than 90% or more than 95% of the fatty acids having between 4 and 8 carbon atoms, and more particularly, between 4 and 6 carbon atoms or between 6 and 8 carbon atoms.

In another particular embodiment, the fractionated oil does not contain any fatty acids having greater than 4 carbon atoms, more particularly, greater than 6 carbon atoms, or even more particularly, greater than 8 carbon atoms.

In one embodiment, the fractionated oil is selected from the group consisting of fractionated coconut oil, fractionated palm oil, fractionated palm kernel oil, fractionated sesame oil, fractionated soybean oil, fractionated almond oil, fractionated rapeseed oil, fractionated corn oil, fractionated sunflower oil, fractionated peanut oil, fractionated olive oil, fractionated castor oil, fractionated soybean oil, fractionated safflower oil, fractionated cottonseed oil, and combinations thereof.

In one embodiment, the nanocarrier comprises fractionated coconut oil. In a particular embodiment, the fractionated coconut oil is enriched for fatty acids having between 4 and 8 carbon atoms and more particularly, between 4 and 6 carbon atoms or between 6 and 8 carbon atoms. In one embodiment, the carrier is fractionated coconut oil having greater than 80%, greater than 85%, greater than 90%, or greater than 95% fatty acids having between 4 and 8 carbon atoms and more particularly, between 6 and 8 carbon atoms. In one embodiment, the carrier is fractionated coconut oil containing butyric, caprioic, caprylic fatty acids, or a combination thereof.

In certain embodiments, the nanocarrier comprises fractionated coconut oil containing greater than 80%, greater than 85%, greater than 90%, or greater than 95% butyric fatty acids.

In certain embodiments, the nanocarrier comprises fractionated coconut oil containing greater than 80%, greater than 85%, greater than 90%, or greater than 95% caprioic fatty acids.

In certain embodiments, the nanocarrier comprises fractionated coconut oil containing greater than 80%, greater than 85%, greater than 90%, or greater than 95% caprylic fatty acids

Fractionated coconut oil is a liquid at room temperature.

In a particular embodiment the nanoformulations contains two or more fractionated oils, e.g., fractionated coconut oil and fractionated palm oil.

In one embodiment, the formulation is an oil-in-water (o/w) formulation and more particularly, an oil-in-water nanoemulsion.

In another embodiment, the formulation is a water-in-oil (w/o) formulation and more particularly, a water-in-oil nanoemulsion. According to this embodiment, the oil phase is the dispersed phase and contains the lipophilic Cannabinoid and fractionated oil.

In a particular embodiment, the fractionated oil is present in an amount between about 0.1% to about 30% w/w, such as from about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, or about 29% to about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w.

The nanoformulation may further comprise one or more additional ingredients. These ingredients may include, for example, anti-oxidants, penetration enhancers, moisturizers, emulsifiers, gelling agents, surfactants, stabilizers, viscosity modifiers, antimicrobial preservatives, irritant-reducing additives, topical anesthetics or combinations thereof.

In one embodiment, the anti-oxidant is selected from the group consisting of butylated hydroxyltoluene (“BHT”), butylated hydroxyl anisole (“BHA”), alpha-tocopherol (Vitamin E), ascorbyl palmitate, ascorbic acid, sodium ascorbate, ethylenediamino tetraacetic acid, cysteine hydrochloride, citric acid, sodium citrate, sodium bisulfate, sodium metabisulfite, lecithin, propyl gallate, sodium sulfate, tert-butylhydroquinone (“TBHQ”) and combinations thereof. In a preferred embodiment, the formulations contain alpha-tocopherol (Vitamin E), ascorbyl palmitate, or combinations thereof

In one embodiment, the penetration enhancer is selected from the group consisting of propylene carbonate, transcutol, pyrrolidones such as N-methylpyrrolidone or N-hydroxyalkylpyrrolidone, azone, menthol, eucalyptol, nicotinamide, glycerol, mono-di- or polyglycols, ethylacetate or Eugenol. The enhanced permeation can be measured, for example, by measuring the rate of diffusion of drug through animal or human skin using a diffusion cell apparatus.

In an emulsion, the penetration enhancer may be in the aqueous phase, the oil phase or both. The amount of the penetration enhancer may vary. In one embodiment, the penetration enhancer is present in an amount between about 0.5 and about 30%, about 1 and about 25%, about 5 and about 20%, or about 10 and about 20%. In certain embodiments, the penetration enhancer is present at about 1%, about 3%, about 5%, about 10%, about 15%, about 20%, or about 25%.

Penetration enhancers were varied between the aqueous and oil phase of the formulations. Based on various formulation, stability and penetration testing results, it has been discovered from the decision matrix, that overall skin penetration potential was optimal for CBD and Cannabinoids with the following penetration enhancers in the given order of significance at the tested optimal concentrations: Transcutol P (15%); propylene glycol (20%); isopropyl myristate (IPM) (4%); isopropyl palmitate (IPP) (4%).

In a particular embodiment, the penetration enhancer is transcutol, more particularly Transcutol® P (D=diethylene glycol monoethyl ether). The transcutol may be present in any suitable amount and more particularly, between about 5 and about 25%, about 10 and about 20%, or about 15%.

In a particular embodiment, the penetration enhancer is propylene glycol. The propylene glycol may be present in any suitable amount and more particularly, between about 10 and about 30%, more particularly about 15 and about 25%, or about 20%.

In a particular embodiment, the penetration enhancer is isopropyl myristate. The isopropyl myristate may be present in any suitable amount, more particularly between about 1 and about 10%, more about 3 and about 5%, or about 4%.

In a particular embodiment, the penetration enhancer is isopropyl palmitate. The isopropyl palmitate is present in any suitable amount, and more particularly, more particularly between about 1 and about 10%, more about 3 and about 5%, or about 4%.

In one embodiment, the moisturizer is selected from the group consisting of glycerin, sorbitol, polyethylene glycols, urea, and propylene glycol.

In one embodiment, the emulsifier is selected from the group consisting of acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

In one embodiment, the stabilizer is selected from the group consisting of albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and combinations thereof.

In addition to additional non-active components, the CBD nanoformulation may contain, in some embodiments, other active materials, such as drugs or agents conventionally used in the treatment of skin diseases, disorders or conditions. Representative, non-limiting agents include antimicrobial agents, anti-inflammatory agents, anti-aging agents and the like.

The nanoformulation may be prepared for administration via various miscellaneous routes, for example, topical administration, transdermal administration, mucosal administration (intranasal, vaginal, etc.) and/or via inhalation.

These nanoformulations can be used in the preparation of individual, single unit dosage form.

The nanoformulation may be prepared for any mode of administration, including oral, sublingual, topical, nasal, rectal, vaginal, parenteral, ophthalmic, otic or the like.

Topical formulations may take the form of an oil, ointment, cream, lotion, patch, balm, salve, liniment, mouse, foam, bar, pencil, emulsion, gel or the like. The nanoformulation can also be incorporated in solid supports selected from the group consisting of hydrogels, wipes, patches and facial masks.

A cream is a viscous liquid or semisolid emulsion of either the oil-in-water or water-in-oil type.

An ointment is a semi-solid preparation which may have a hydrocarbon bases (oleaginous), adsorption bases (anhydrous); emulsion bases (water and oil type); and water soluble bases. They are more moisturizing and more occlusive than creams, and form a protective film over the skin.

A lotion is a liquid or semi-liquid preparation that contains one or more active ingredients in an appropriate vehicle. A lotion may be a suspension of solids in an aqueous medium, an emulsion, or a solution.

A gel is a sticky, jelly-like semisolid or solid prepared from high molecular weight polymers in an aqueous or alcoholic base. Alcoholic gels are drying and cooling, and are best suited for acute exudative pruritic eruptions; non-alcoholic gels are more lubricating and are well suited, for example, to dry scaling lesions.

The nanoemulsions disclosed herein may be optically transparent or faintly turbid. Their opacity may be expressed in terms of turbidity (τ) and characterized by transmission measurements.

In one embodiment, the nanoformulation is a beneficial cosmetic preparation which may be used in preventing, managing, or treating various skin conditions, e.g., aging skin.

In a particular embodiment, the nanoformulation exhibits low irritation potential when measured in vitro.

In another particular embodiment, the nanoformulation exhibits low irritation potential when measured in vivo.

The nanoformulation disclosed herein are advantageously stable for up to about two years and in certain embodiments, greater than about two years. Stability may be measured by any suitable means. In a particular embodiment, the means for assessing stability is selected from centrifuge and thermal stress analyses. The analysis measurement may also vary. In certain embodiments, the analysis measurements are selected from droplet size, pH value and electrical conductivity.

In certain embodiments, droplet size analysis of nanoemulsion is measured by a diffusion method using a light-scattering, particle size-analyzer counter, photon correlation spectroscopy, or transmission electron microscopy (TEM).

In a certain embodiment, the nanoformulations disclosed herein are stable at about 40° C. about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment, the nanoformulations disclosed herein are stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

In other embodiments, the nanoformulations disclosed herein are stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

In a particular embodiment, the nanoformulation disclosed herein is kinetically stable at ambient or room temperature.

In another particular embodiment, the nanoformulation disclosed herein is thermally stable at ambient temperature.

Kits that include the nanoformulation of the present invention are also contemplated. In certain embodiments, the nanoformulation (or components thereof) is comprised in a container. The container can be a bottle, dispenser, or package. The container can dispense a pre-determined amount of the nanoformulation. The container can include indicia on its surface. The indicia can be a word, an abbreviation, a picture, or a symbol.

In one embodiment, the nanoemulsion comprises water, at least one fractionated oil and at least one cannabinoid, and at least one penetration enhancer, wherein the at least one fractionated oil is fractionated coconut oil; the at least one cannabinoid is CBD; and wherein the penetration enhancer is selected from diethylene glycol monoethyl ether, propylene glycol, isopropyl myristate, isopropyl palmitate or combinations thereof. In certain embodiments, the CBD is a component of a nanoparticle between about 40 and about 50 nM and the nanoemulsion is absorbed by the skin within a matter of minutes and in particular, less than about 5 minutes, less than about 3 minutes, less than about 1 minutes, less than about 50 seconds, less than about 40 second or less than about 30 seconds.

In another embodiment, the nanoemulsion comprises water, at least one fractionated oil and at least one cannabinoid, and at least one penetration enhancer, wherein the at least one fractionated oil is short-chained fractionated coconut oil; the at least one cannabinoid is CBD; and wherein the penetration enhancer is selected from diethylene glycol monoethyl ether, propylene glycol, isopropyl myristate, isopropyl palmitate or combinations thereof. In certain embodiments, the CBD is a component of a nanoparticle between about 40 and about 50 nM and the nanoemulsion is absorbed by the skin within a matter of minutes and in particular, less than about 5 minutes, less than about 3 minutes, less than about 1 minutes, less than about 50 seconds, less than about 40 second or less than about 30 seconds.

In another embodiment, the nanoemulsion comprises water, at least one fractionated oil and at least one cannabinoid, and at least one penetration enhancer, wherein the at least one fractionated oil is short-chained fractionated coconut oil; the at least one cannaboid is CBD; and wherein the penetration enhancer is diethylene glycol monoethyl ether, e.g., (Transcutol P®). In certain embodiments, the CBD is a component of a nanoparticle between about 40 and about 50 nM and the nanoemulsion is absorbed by the skin within a matter of minutes and in particular, less than about 5 minutes, less than about 3 minutes, less than about 1 minutes, less than about 50 seconds, less than about 40 second or less than about 30 seconds.

In certain embodiments, the nanoemulsion is odorless, tasteless or both. In one embodiment, the nanoemulsion does not contain any preservatives.

### III. Methods of Use

Disclosed herein are methods of using the nanoformulation of CBD disclosed herein, such as methods of treating a skin disease, disorder or condition. Currently, most formulation of CBD are limiting in therapeutic efficacy, since most are at a sub-therapeutic dose, secondary to concentration, formulation, purity or delivery method. The current technology and formulation aims to change these factors to optimize the delivery and concentration for fast acting efficacy, while still within the safety standards.

In one embodiment, the method comprising administering a therapeutically effective amount of the CBD nanoformulation disclosed herein to a subject in need thereof, thereby treating a skin disease, disorder or condition. The skin disease, disorder or condition may be acute or chronic. In certain embodiments, the subject is suffering from two or more diseases, disorders or conditions of the skin.

In certain embodiments, treatment according to the method disclosed herein results in a reduction in one or more symptoms of the skin disease, disorder or condition. The reduction in one or more symptoms may be clinically observed, clinically reported or both.

In certain embodiments, the reduction in symptoms relates to a symptom selected from one or more of the following: redness, heat/warmth, discomfort (e.g., itching, burning, stinging), pain, swelling, number of lesions, scales, rashes or the like. In certain embodiments, the reduction in one or more symptoms is clinically observed, clinically reported or both relative to baseline for the subject. The reduction may be, for example, between about 0.1 to about 100%, or about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the reduction is not associated with any adverse events.

In a particular embodiment, a subject treated according to the method disclosed herein exhibits a reduction in erythema, scale, lesion count and/or local skin reactions compared to baseline. The reduction may be, for example, between about 0.1 to about 100%, or about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%. In certain embodiments, the reduction is not associated with any adverse events.

In a particular embodiment, a subject treated according to the method disclosed herein exhibits an improvement in investor gross analysis (IGA) compared to baseline. In certain embodiments, the result of treatment is that the patient has an IGA score of 0 (clear), 1 (almost clear) or a ≥2-point improvement. In certain embodiments, the improvement is not associated with any adverse events. In certain embodiments, the method is carried out over a population of patients and at least a majority (50% or more) of subjects exhibit an improvement in their IGA score, e.g., the result of treatment is that the majority of treated subject have an IGA score of 0 (clear), 1 (almost clear) or a ≥2-point improvement.

In another particular embodiment, a subject treated accorded to the method disclosed herein exhibits an improvement (reduction) on a Eczema Area and Severity Index (EAST). The EASI scoring assessment multiplies the percentage of the affected area (of four specific skin regions) and then adds this number to the severity scores of the four specific symptoms. In certain embodiments, the subject's EASI is decreased by about 1 or about 2 points. In a particular embodiment, the method is carried out over a population of patients and at least the majority (50% or more) of subject exhibit an improvement (reduction) in their EASI.

In a particular embodiment, a subjected treated according to the method disclosed herein exhibits a result that is superior to a subject treated with Pimecrolimus 1% cream.

In certain embodiments, the therapeutic effect of the method disclosed herein is observed more quickly than the therapeutic effect of formulations of CBD known in the art, and in particular, non-nano formulations of CBD. In one embodiment, the therapeutic effect (e.g, the reduction in one or more symptoms, such as erythema or pruritic symptoms) is evident within about 30 minutes, within about 25 minutes, within about 20 minutes, within about 15 minutes, without about 10 minutes, or within about 5 minutes or less.

In a particular embodiment, the therapeutic effect (e.g., the reduction in one or more symptoms) is evident within about 5 to about 20 minutes, within about 10 and about 15 minutes or about 15 minutes. The fact that the therapeutic effect is evident within a certain time frame does not imply that the therapeutic effect may not continue to develop or increase. For example, the subject's skin may continue to improve or clear over a period of hours or days.

In certain embodiments, the subject treated according to the method disclosed herein had previously been treated one or more times with an agent(s) conventionally used in treatment of a skin disease, condition or disorder suffered by the subject but with limited or insufficient results. The method disclosed herein advantageously improves on that prior treatment (s), by clinical observation, clinical reporting or both. In certain embodiments, the improvement is statistically significant compared to baseline.

In embodiments where the therapeutic effect is a reduction in pain or pain control, the therapeutic effect is observable relatively quickly, i.e., within one hour, more particularly, within about 30 minutes and even within about 15 to about 20 minutes, and is maintained or increases over a period of time thereafter on the order of hours, i.e., about 1 to about 5 hours, about 2 to about 3 hours. Advantageously, the formulation permits treatment or management of pain without the potential for addiction. In certain embodiments, the nanoformulation disclosed herein is useful for treatment of even high-impact chronic pain.

The most suitable route of administration will depend on the nature and severity of the condition being treated. In one embodiment, the administration is topical. In another, the administration is ophthalmic, sub-lingual, oral or systemic.

The CBD nanoformulation may be applied topically to any convenient topical site. Representative, non-limiting topical sites include the face, neck, arms, legs, and torso. In one embodiment, the CBD nanoformulation is applied to the face, where the application is to the entire face or partial face or the particular lesion. In one embodiment, the CBD nanoformulation is typically applied to a selected localized region that is less than 10% of the total surface area of the subject. In certain embodiments, the localized region is less than 5% of the total surface area of the subject.

In a particular embodiment, the CBD nanoformulation is applied topically to a selected area of the skin of a human subject and in particular, a selected area of the face.

In a particular embodiment, the CBD nanoformulation is applied topically to a selected area of the skin of a human subject and in particular, a selected area of the extremities, such as the arm or legs.

A cream, lotion, gel, ointment, spray, foam, paste or the like may be spread on the affected surface and gently rubbed in. A solution may be applied in the same way, but more typically will be applied with a dropper, swab, or the like, and carefully applied to the affected area.

Disorders of the skin include a wide variety of pathologies, sometimes associated with an underlying disease state.

In one embodiment, the skin disorder is a non-neoplastic skin disorders, such as an inflammatory skin disorder. Such disorders may be acute or chronic. In certain instances, the inflammatory skin condition may be immune-mediated. In some embodiments, the subject treated according to the methods disclosed herein have shown a limited or inadequate response to conventional therapies for the skin disease or disorders and continue to clinically report symptoms of the disease or disorder.

Clinical signs (symptoms) associated with inflammatory skin disorders include redness, edema, excoriation, hair loss, ulceration, lichenification, pruritus, and dry, flaky skin.

In one embodiment, the method of the present invention useful for treatment of an inflammatory skin disorder such as atopic dermatitis (AD), granulomatous dermatitis (GD), pityriasis lichenoides (PL), psoriasiform dermatitis (PD), panniculitis or spongiotic dermatitis (SD) and other papulosquamous and eczematous dermatoses such as psoriasis, erythroderma, lichen planus, lichenoid dermatoses, small/large plaque parapsoriasis, pityriasis lichenoides, pityriasis rubra pilaris, pityriasis rosea, pityriasis rotunda, and granular parakeratosis.

In certain embodiments, the skin disease or disorder is psoriasis A common genetically determined, chronic, inflammatory skin disease characterized by rounded erythematous, dry, scaling patches. The lesions have a predilection for nails, scalp, genitalia, extensor surfaces, and the lumbosacral region. Accelerated epidermopoiesis is considered to be the fundamental pathologic feature in psoriasis.

In another embodiment, the method of the present invention is useful for treatment of an inflammatory skin disorder such as other eczematous eruptions including: seborrheic dermatitis, asteatotic eczema, stasis dermatitis; disseminated eczema, nummular dermatitis, periorificial eczema dermatitis, pityriasis alba, infective dermatitis, dyshidrotic eczema, juvenile plantar dermatosis and diaper dermatitis.

In another embodiment, the method of present invention is useful for treatment of an inflammatory skin disorder such as urticarias, erythemas and purpuras. Further subclassified as: urticaria, angioedema, figurate erythemas, erythema multiforme, steven-johnson syndrome, toxic epidermal necrolysis, drug reaction urticarias and erythemas, purpura, cutaneous vasculitis and eosinophilic/neutrophilic/pregnancy dermatoses.

In another embodiment, the method of present invention is useful for treatment of an inflammatory skin disorder of rheumatologic origin. These include: autoimmune connective tissue diseases, lupus erythematosus, dermatomyositis, systemic sclerosis (scleroderma), morphea, lichen sclerosus, still's disease, relapsing polychondritis, sjogren's syndrome, mixed connective tissue disease, extra-articular manifestations of rheumatoid arthritis, interstitial granulomatous dermatitis, palisaded neutrophilic and granulomatous dermatitis.

In another embodiment, the skin disease or disorder is rosacea, common acne, inflammatory acne, orrheic dermatitis, eczema psoriasis (e.g., (plaque psoriasis), rashes, itch, perioral dermatitis, acneform rashes, transient acantholytic dermatosis, acne necrotica miliaris, pruritus, dysesthesia and psychocutaneous diseases.

In certain embodiments, the skin disease or disorder is acne vulgaris. As an ordinary artisan would understand, acne vulgaris is chronic disorder of the pilosebaceous apparatus associated with an increase in sebum secretion. It is characterized by open comedones (blackheads), closed comedones (whiteheads), and pustular nodules.

In certain embodiments, the skin disease or disorder is rosacea. As an ordinary artisan would understand, rosacea is an acneiform eruption occurring mostly in middle-aged adults and appearing generally on the forehead, cheeks, nose, and chin. Three types are recognized: granulomatous, glandular hyperplastic with rhinophyma, and ocular.

In another embodiment, the skin disease or disorder is atopic dermatitis, eczema, pruritus and itch, pain, psoriasis, acne, scleroderma, skin cancers, rosacea and erythema, vitiligo and inflammatory pigment disorders, neutrophil diseases, dermatomyositis, hidradenitis suppurativa, lichen simplex chronicus, prurigo nodularis, cutaneous lupus erythematosus (CLE), inflammation secondary to infections, infestations and bites, and cosmetic uses secondary to inflammation.

In certain embodiments, the skin disease or disorder is atopic dermatitis. As an ordinary artisan would understand, atopic dermatitis is a chronic inflammatory genetically determined disease of the skin marked by increased ability to form reagin (IgE), with increased susceptibility to allergic rhinitis and asthma, and hereditary disposition to a lowered threshold for pruritus. It is manifested by lichenification, excoriation, and crusting, mainly on the flexural surfaces of the elbow and knee. In infants it is known as infantile eczema.

In certain embodiments, the disease or disorder is eczema. As an ordinary artisan would understand, eczema is characterized by initial symptoms of blistering and dry cracked skin on the hands or feet, affecting the tips and sides of fingers, toes, soles and palms. The disease develops progressively into continual scaling, peeling, cracked skin, bleeding, deep fissures and open wounds. These conditions cause the skin to become red, itchy and inflamed. There are several types of eczema: atopic dermatitis, contact dermatitis, dyshidrotic eczema, nummular eczema, seborrheic dermatitis and stasis dermatitis.

In certain embodiments, the skin disease or disorder is shingles. As an ordinary artisan would understand, shingles refers to an outbreak of rash or blisters on the skin caused by varicella-zoster virus (VZV). The first sign of shingles is often burning or tingling pain, or sometimes numbness or itch, in one particular location on only one side of the body. After several days or a week or more, of nerve pain, a rash of fluid-filled blisters, similar to chickenpox, appear in one area on one side of the body. Shingles pain can be mild or intense and may persist with debilitating effect.

In certain embodiments, the skin disorder is postherpetic neuralgia. As an ordinary artisan would understand, post herpetic neuralgia is a frequent adverse event in herpes zoster patients.

In certain embodiments, the skin disorder is a regional pain syndrome. As an ordinary artisan would understand, regional pain syndrome is a chronic pain condition in which high levels of nerve impulses are sent to an affected site.

In certain embodiments, the skin disorder is a scar(s). As an ordinary artisan would understand, scars are classified into different categories, based on the nature of the injury having caused the scar, its clinical characteristics and its appearance. In a particular embodiment, the scar is a flat or pale scale, a sunken scar, a hypertrophic scar or a keloids scar.

In certain embodiments, the skin disorder is pigmentation.

In a particular embodiment, the skin disease is a hyperplasia, i.e., a proliferation of cells which is reactive and not neoplastic.

In a particular embodiment, the disease is a dysplasia, i.e., an atypical proliferation of cells intermediate between hyperplasia and neoplasia.

In another embodiment, the skin disease is a neoplastic disease (e.g., cancer). A neoplasm may be benign or malignant. Malignancy is generally associated with 1) local invasion, in which the neoplasm extends into vital organs and interferes with their function, and/or 2) metastasis, in which cells from the tumor seed out to other parts of the body and then grow into tumors themselves.

In certain embodiments, the damage to the skin resulting from any type of cancer treatment (e.g., chemotherapy, radiotherapy, surgery, immunotherapy including bone marrow or hematopoietic grafting, GVHD, etc.).

In one embodiment, the disease or disorder treated by the method disclosed herein is a carcinoma, i.e., a malignant neoplasm with cells that appear to be derived from epithelium.

In one embodiment, the disease or disorder treated by the method disclosed herein is a sarcoma, i.e., a malignant neoplasm with cells that appear be derived from those other than epithelium, e.g., derived from connective tissue.

Examples of cancers of the skin include, for example, basal cell carcinoma, squamous cell carcinoma, malignant melanoma, Bowen's disease, Kaposi's sarcoma, dermatofibrosarcoma, Merkel cell carcinoma, and Paget's disease of the nipple.

In certain embodiments, the skin disease or disorder is a basal cell carcinoma. As an ordinary artisan would understand, basal cell carcinoma is a malignant skin neoplasm that seldom metastasizes but has potentialities for local invasion and destruction. Clinically it is divided into types: nodular, cicatricial, morphaic, and erythematoid (pagetoid). More than 95% of these carcinomas occur in patients over 40. They develop on hairbearing skin, most commonly on unexposed areas. Approximately 85% are found on the head and neck area and the remaining 15% on the trunk and limbs.

In certain embodiments, the skin disease or disorder is melanoma. As an ordinary artisan would understand, a melanoma is a tumor arising from the melanocytic system of the skin and other organs. When used alone, the term refers to malignant melanoma. It occurs mostly in adults and may originate de novo or from a pigmented nevus or malignant lentigo.

In certain embodiments, a method is disclosed for using the composition disclosed herein for treating pain in a subject in need thereof. The pain may be acute or chronic. The pain may be manifest as pain associated with the skin, either under normal circumstances or in response to a stimulus such as heat, light or pressure (e.g., touch). The pain may result from a disorder of the skin or an underlying disorder (e.g., an infection).

Dosages and dosing frequency will be determined by a trained medical professional depending on the activity of the compounds used, the characteristics of the particular topical formulation, and the identity and severity of the dermatologic disorder treated or prevented.

In one embodiment, the nanoformulation is administered on a regular basis.

In a particular embodiment, the nanoformulation applied once or twice per day or more, depending on the severity of the condition. In a particular embodiment, the nanoformulation is administered once per day (QD). In another particular embodiment, the nanoformulation is administered twice per day (BID).

In another embodiment, the nanoformulation is applied once or twice per week or more, depending on the severity of the condition.

In certain embodiments, the nanoformulation is administered once or twice per day over a period of time that may vary, e.g., about 1 week, about 2 weeks, about 3 weeks, about 4 weeks, about 5 weeks, about six weeks, about eight weeks, about 2 months, about 3 months.

In one embodiment, the in vitro penetration of the CBD nanoformulation disclosed herein is improved relative to conventional CBD formulations. Various in vitro models of percutaneous penetration are known in the art, including, without limitation, diffusion cell studies, attenuated total-reflectance-Fourier transform infrared spectroscopy, and tape stripping. Penetration can be measured by any suitable technique, e.g., by TIR spectra and DSC thermogram.

In a particular embodiment, penetration is measured using transport across cell monolayers.

In a particular embodiment, the in vitro penetration of the CBD nanoformulation disclosed herein is improved by about 5% to about 50%, compared to a conventional topical CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In another embodiment, the in vivo penetration of the CBD nanoformulation disclosed herein is improved relative to conventional CBD formulations. The in vivo penetration may be in an animal (e.g., mouse) model or a human patient.

In a particular embodiment, the in vivo penetration of the CBD nanoformulation disclosed herein is improved by about 5% to about 50%, compared to a conventional topical CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In a particular embodiment, the CBD nanoformulation disclosed herein has an increased permeability coefficient, kip, relative to a conventional topical CBD formulation. In a particular embodiment, the permeability coefficient is increased by about 50% or more.

In one embodiment, the CBD nanoformulation disclosed herein has superior absorption compared to non-nano CBD formulations known in the art. In a particular embodiment, the CBD nanoformulation is absorbed by the skin in less than sixty minutes, less than forty minutes, less than thirty minutes, less than twenty minutes, less than fifteen minutes, less than ten minutes, less than five minutes, less than 4 minutes, less than 3 minutes, less than 2 minutes or less than one minute but in each case is absorbed.

In another embodiment, the CBD nanoformulation disclosed herein has superior bioequivalency compared to non-nano CBD formulation known in the art. In a particular embodiment, the CBD nanoformulation disclosed herein has a bioequivalency greater than about 15%, greater than about 20%, greater than about 25%, greater than about 30%, greater than about 35%, greater than about 40%, greater than about 45%, greater than about 50%, greater than about 55%, greater than about 60, greater than about 65%, greater than about 70%, greater than about 75%, greater than about 80% or greater than about 85%.

In another embodiment, the CBD nanoformulation has a bioavailability of greater than about 10%, greater than about 15%, greater than about 20%, greater than about 25%, greater than about 30%, greater than about 35%, greater than about 40%, greater than about 45% or greater than about 50% or more.

In a particular embodiment, the CBD nanoformulation has a bioavailability of between about 15% and about 50%, about 20% and about 45%, about 25% and about 40% or about 30%.

In another embodiment, the CBD nanoformulation disclosed an improved bioavailability of about 5% to about 50%, compared to a conventional topical CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In a particular embodiment, the CBD nanoformulation disclosed herein has an increase in maximum steady state flux, Jmax, relative to a conventional topical CBD formulation. In a particular embodiment, the maximum steady state flux is increased by about 25% or more.

In certain embodiments, the methods disclosed result in a reduction in the number of skin lesions.

In one embodiment, the methods disclosed herein permit an improvement in one or more patient reported outcomes (PRO).

In a particular embodiment, the methods disclosed herein permit an improvement in the subject's quality of life (QOL). The quality of life may be measured by any suitable instrument, including, for example, the Dermatology Life Quality Index. (Finlay A Y, et al., Clin Exp Dermatol. 1994; 19:210-6).

### IV. Method of Manufacture

Cannabinoids exhibit poor water solubility, which complicates their delivery to the blood stream and reduces the associated bioavailability. Particle size reduction to the nanometer range of these substances increases their aqueous dissolution rate and solubility, which results in improved bioavailability, accelerated onset of action, and decreased potential of harmful side-effects.

The lipid-based nanoparticles disclosed herein may be produced by any suitable method. In one embodiment, the lipid-based nanoparticle is produced by a method selected from top down methods, bottom up methods or a combination thereof.

In a particular embodiment, the lipid-based nanoparticle is prepared by a method selected from high pressure homogenization (e.g., hot or cold high-pressure homogenization), double emulsion, microemulsion, ultrasonication, solvent evaporation, solvent emulsification-diffusion, super critical fluid methods, spray drying or combinations thereof.

In a particular embodiment, the lipid-based nanoparticle is a liposome and the liposome is produced by dissolving the cannabidiol in an organic solvent, then mixing the same with lipids dissolved in a miscible organic solvent. The thin lipid film produced by rotary evaporation is then hydrated by adding an aqueous solution. The resultant multilamellar liposomes are extruded through membranes with defined pore size or sonicated to form small unilamellar vesicles of desired size.

In a particular embodiment, the lipid-based nanoparticle is made from high pressure homogenization (HPH). The term “homogenization” refers to the production of a homogeneous size distribution of particles suspended in a liquid, by forcing the liquid under the effect of pressure through a specifically designed homogenization valve. According to this technique, the cannabidiol is first solubilized in the melted lipid.

In another particular embodiment, the lipid-based nanoparticle is made by ultrasonication. In a particular embodiment, the method is probe ultrasonification (i.e., direct or indirect). The process of ultrasonic top-down nanocrystallization requires extremely high ultrasonic amplitudes to be applied to particle suspensions producing extreme shear forces. The shear forces are the result of intense ultrasonic cavitation, which creates imploding vacuum bubbles and causes micro-jets that break up the original drug particles down to the nano-size range.

The ultrasonication settings comprise one or more of an amplitude, a frequency, a power, and a duration. For example, in some embodiments, the ultrasonication setting includes an amplitude between 25 and 100 microns. In certain embodiments, the ultrasonication is applied at a frequency in the range of 20 Hz to 20 kHz. In some embodiments, the ultrasonication is applied at a frequency around 200 Hz, e.g., in the range of 175 to 225 Hz. In some embodiments, the ultrasonication applied at a power in the range from 100 to 400 W. In some embodiments, the ultrasonication is applied for a particular duration, such as for 15 minutes or longer.

In a particular embodiment, ultrasonic amplitudes of at least 70 microns are used to take full advantage of ultrasonic cavitation.

In another embodiment, the nanoparticles are made to an average size of 50 nms for enhanced penetration and bioavailability.

In certain embodiments, the lipid-based nanoparticle composition comprises cannabidiol that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have cannabidiol in them.

Characteristics such as loading capacity, drug release rate, physical and chemical stability, and vesicle size depend on experimental conditions, and material and method choices at the time of preparation, as would be understood by one of skill in the art.

### EXAMPLES

### Example 1: Lipid-Based Nanoparticles

A fractionated coconut oil is provided containing caproic acid (C6) and caprylic (C8) acid, but substantially no fatty acids having more than 8 carbon atoms.

The fractionated coconut oil is used to form a lipid-based nanoparticle.

The lipid-based nanoparticle is then loaded with cannabidiol to provide a nanoparticle formulation.

### Example 2: HPLC Analysis

The HPLC analytical method, selected based on understanding of the literature, for the detection/quantification of CBD and its related substances is detailed in Table 1.

TABLE 1
HPLC method for detection/quantification of CBD and related substances.

| HPLC System | Shimadzu Prominence LC with Diode-Array Detection | | |
|---|---|---|---|
| | Shimadzu LabSolutions Chromatography Software | | |
| Column | Waters XBridge Phenyl Column 4.6 × 150 mm [symbol] 3.5 μm | | |
| Guard Column | Waters XBridge BEH Phenyl 3.9 × 5 mm [symbol] 3.5 μm | | |
| Mobile phase A | 100% Water | | |
| Mobile phase B | 100% Acetoniltrile | | |
| Initial flow rate | 1.0 mL/min | | |
| Run time | 35 minutes | | |
| Wavelength | 235 nm | | |
| Column temperature | 40° C. | | |
| Auto sampler temperature | 10° C. | | |
| Flow gradient | Time | % Mobile phase A | % Mobile phase B |
| | 0.00 | 95.0 | 5.0 |
| | 5.0 | 95.0 | 5.0 |
| | 12.00 | 40.0 | 60.0 |
| | 25.00 | 34.0 | 66.0 |
| | 27.00 | 10.0 | 90.0 |
| | 30.00 | 10.0 | 90.0 |
| | 30.01 | 95.0 | 5.0 |
| | 35.00 | 95.0 | 5.0 |

| | |
|---|---|
| Injection volumn | 35 μL |
| Retention time of CBD | ca. 19.65 min |
| Sample and standard diluent | 100% Methanol |
| Seal wash | 20% 2-Propanol in Water |
| Needle Rinse | 60 [symbol] 40 v/v Methanol [symbol] Water |
| Range | 25.0-150 μg/mL |

[symbol]

indicates data missing or illegible when filed

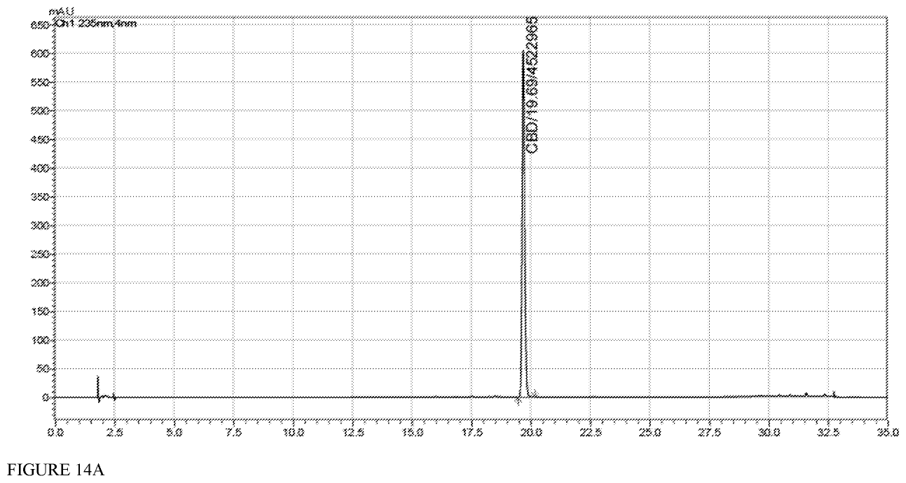

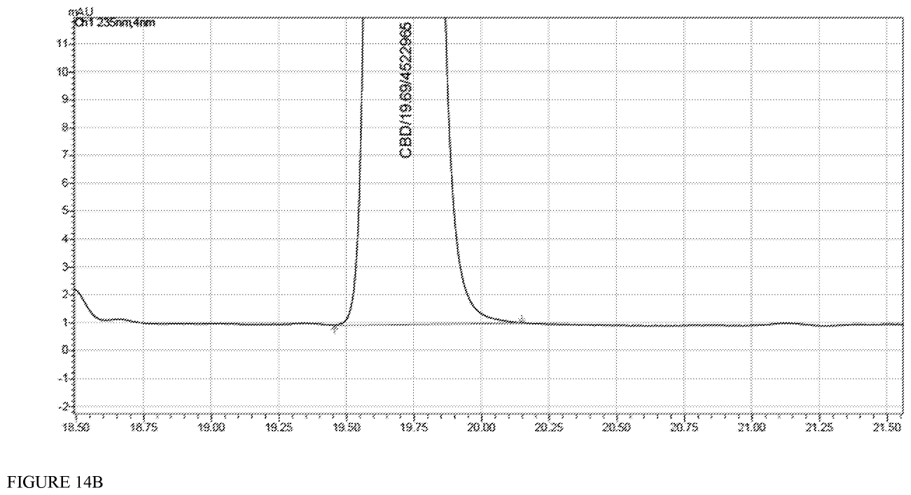

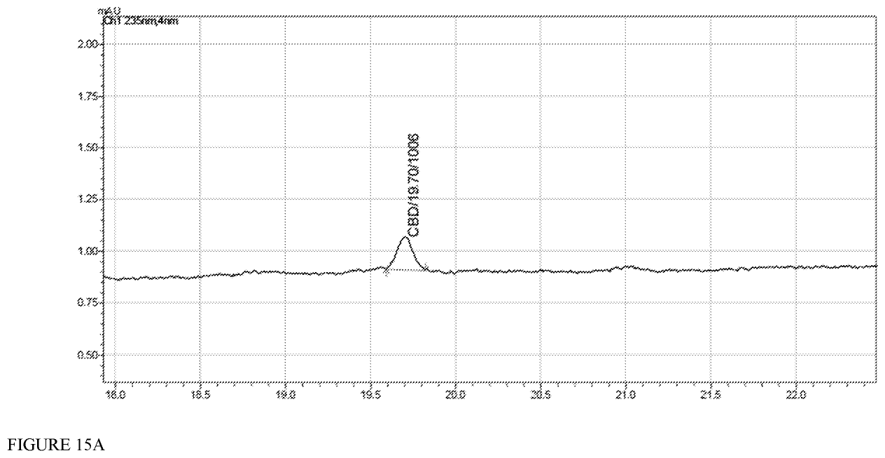

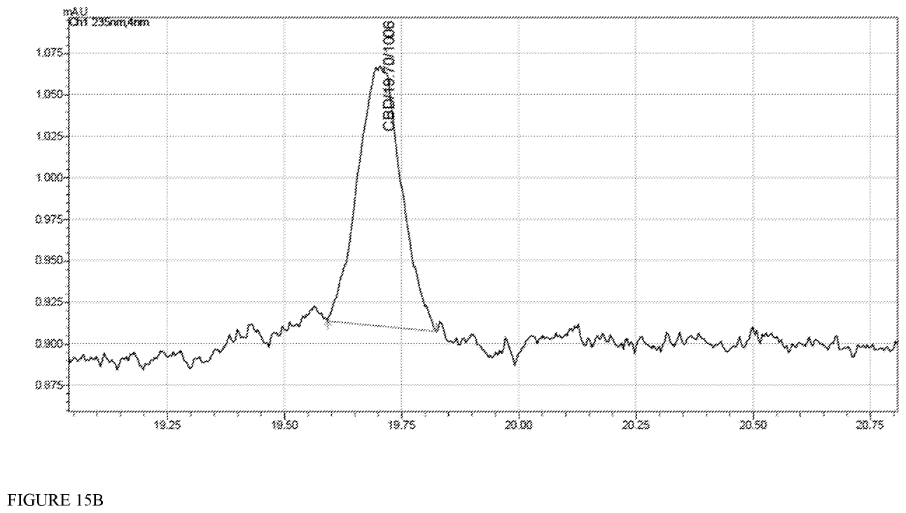

This method was assessed and subsequently used for analysis of formulation stability samples. Calibration standards of CBD over a concentration range of 25.0-150 μg/mL were prepared, and a representative calibration plot is presented in FIG. 14.

### Example 3: Clinical Observations

The initial clinical cases utilizing the compositions disclosed herein provided strong evidence of its penetration potential and associated anti-inflammatory efficacy parameters in the skin. Various formulations have been tested on multiple skin inflammatory conditions with high efficacy and safety results as outlined in the following case reports. The observational anti-erythematic, inflammatory or pain responses are relatively fast and in line with the quick absorption and penetration paradigm. Over 100 cases have been studied for multiple inflammatory skin and pain conditions. A summary of the case findings is outlined in Table 2:

CLINICAL OBSERVATIONS & TESTIMONIALS

| | | | | | | | Regional | |
|---|---|---|---|---|---|---|---|---|
| Disease or | | | Inflammatory | | | Other | Pain | |
| Symptoms | Eczema | Psoriasis | Acne | Rash/Itch | Hives | Dermatitis | Syndrome | Cosmetic |
| Positive | 18 | 4 | 5 | 23 | | 7 | 18 | 10 |
| Response | | | | | | | | |
| Minimal | 2 | 2 | 1 | 2 | 1 | 1 | 2 | |
| Response | | | | | | | | |

### Example 4: Clinical Study in Atopic Dermatitis

A clinical study is carried out for subjects with atopic dermatitis as describe in Table 3, below:

TABLE 3
Atopic Dermatitis

| | |
|---|---|
| Title | A Single Center, Open-Label Study of the Safety and Efficacy of Canno |
| | Cream in Subjects with Atopic Dermatitis |
| Study Type | Proof of Concept |
| Test Articles | 1. Canno Cream (Greenway Therapeutix_{@}) |
| Study Objective | To determine the safety and efficacy of Canno Cream in subjects with |
| | Seborrheic Dermatitis |
| Study Design | Single open-label study |
| Treatment Groups | After enrollment in the study, each subject will be assigned test article will |
| | be applied for 4 (four) weeks. |
| Duration of Treatment | Four weeks |
| Duration of Study | Four weeks |
| Study Population | Male or female subjects aged 18 years and older with Seborrheic Dermatitis |
| | of the face |
| Total Number of | Approximately 12 subjects will be enrolled to obtain about 12 evaluable |
| Subjects | subjects in the study. |
| Number of Sites | One site will participate in the study. |
| Inclusion Criteria | To enter the study, a subject must meet the following criteria: |
| | 1. Subject is a male or female, 18 years of age or older. |
| | 2. Subject is willing and able to give written informed consent. |
| | 3. Subject is willing and able to apply Canno Cream as directed, comply |
| | with study instructions and commit to all follow-up visits for the |
| | duration of the study. |
| | 4. Subject has a clinical diagnosis of Seborrheic Dermatitis of at least |
| | mild severity on the face. The presence or absence of Seborrheic |
| | Dermatitis on the scalp, chest, or other body surface area is not |
| | significant but can be treated using the test article upon consent of the |
| | investigator and subject. |
| | 5. Subject is in good general health and free of any disease state or |
| | physical condition that might impair evaluation of Seborrheic |
| | Dermatitis or which, in the investigator’s opinion, exposes the subject |
| | to an unacceptable risk by study participation. |
| | 6. Females must be post-menopausal¹, surgically sterile² or use an |
| | effective (SEE NOTE AT END OF SYNOPSIS) method of birth |
| | control³,⁴ with a negative urine pregnancy test (UPT)⁵ at the Baseline |
| | Visit. |
| Exclusion Criteria | A subject is ineligible to enter the study if he/she meets one or more of the |
| | following criteria: |
| | 1. Female subject who is pregnant, lactating or planning a pregnancy |
| | during the study period. |
| | 2. Subject is currently enrolled in an investigational drug or device |
| | study. |
| | 3. Subject has used topical medications dedicated to the treatment of |
| | seborrheic dermatitis containing steroids, calcineurin inhibitors, |
| | crisaborole ointment, antifungal agents, or any other treatments |
| | containing CBD within one week of Baseline. |
| | 4. Subject has used recreational marijuana or any other form of |
| | cannabis, cannabinoids, or similar products within 2 weeks of |
| | baseline visit. |
| | 5. Subject has any skin or medical condition, including facial hair, |
| | which could interfere with the evaluation of the study drugs. |
| | 6. Subjects who have significant neurological conditions (Parkinson’s |
| | disease or Stroke), who in the opinion of the investigator are not |
| | eligible for the study due to the severity of neurological condition |
| | to determine severity or ability to complete of trial. |
| | 7. Subject have any history of overt bacterial, viral or fungal infection |
| | of the head/neck. |
| | 8. Subject has any presence of compromising dermatosis elsewhere |
| | on the skin of the face |
| | 9. Subject has excessively actinically damaged skin in the opinion of |
| | the investigator or history of NMSC of the face within 6 months. |
| | 10. Subject plans to be exposed to artificial tanning devices or |
| | excessive sunlight during the study. |
| | 11. Light treatments, microdermabrasion or chemical peels to the face, |
| | chest and back within four (4) weeks of Baseline. |
| | 12. Subject has a history of alcohol and/or drug abuse or is unreliable |
| | in the investigator's judgment. |
| | 13. Subject is known to be hypersensitive to any of the study drug(s) or |
| | any components in the study drug(s). |
| Study Procedures | Subjects can be screened for the study up to 30 days before Visit 1. During |
| | screening, the study requirements will be reviewed, written informed consent |
| | obtained and eligibility confirmed. If applicable, the washout from |
| | prohibited medications or treatments will be determined and implemented. |
| | These procedures may be combined with the Baseline Visit. |
| | Visit 1 (Baseline Visit): At the visit, the study staff will explain the study |
| | procedures and an informed consent must be signed prior to the initiation |
| | of any study-related procedures. At this visit, consenting subjects will have |
| | their medical history, facial dermatologic exam, and inclusion/exclusion |
| | (I/E) criteria reviewed to determine subject eligibility. A urine pregnancy |
| | test [UPT] (if applicable), Fitzpatrick Skin Type assessment, clinical |
| | evaluations (erythema, scale, IGA, LSRs on the face) will be performed at |
| | this visit. A grade of 0 or 1 (clear or almost clear, respectively) on the IGA |
| | scale would be considered a treatment success. |
| | Prior and concomitant therapy and concomitant medications/procedures |
| | will be reviewed. Subjects who require a “washout” period for longer than |
| | 30 days prior to baseline will be re-consented. |
| | Photographs prior to initiation of test article will be taken using of the face. |
| | These photos will only be used for the purpose of the study unless further |
| | consent for use of marketing the approved product at a later date is |
| | approved. |
| | The subject’s face will be free of any makeup, moisturizer, sunscreen, or |
| | any other topical agent on Visit 1 (Baseline) Day 1. Male subjects will be |
| | clean shaven. The designated treatment area will be cleansed with an |
| | approved facial cleanser and allowed to dry for 5 minutes prior to |
| | application of the test article. |
| | At Visit 1 (Baseline) Day 1, the test article will be applied by the subject in |
| | the clinic after clinical evaluations (lesions counts, IGA) and the severity of |
| | any local skin reactions (LSRs) are completed for the face. Subjects will |
| | be instructed on the proper application of the study medication. |
| | The first dose of medication will be applied in the clinic with the assistance |
| | of the clinic staff. After 10 minutes from the first application, a tolerability |
| | assessment for any LSRs will be performed. |
| | One canister of Canno Cream will be dispensed to the subject. One pump |
| | from the canister will provide enough cream to cover the face. One pump |
| | will be used each treatment day. The subject’s existing regimen of |
| | moisturizers and cleansers will be allowed but other treatments that may |
| | impact the immune mechanisms and response will not be allowed during |
| | the treatment phase. |
| | LSRs including burning, stinging, dryness, and itching will be assessed by |
| | the investigator using a five-point ordinal scale (0 = none, 1 = trace, 2 = |
| | mild, 3 = moderate, and 4 = severe) and erythema, edema and |
| | scaling/dryness will also be assessed using a five-point ordinal scale (0 = |
| | none, 1 = minimal, 2 = mild, 3 = moderate, and 4 = severe). Subjects will |
| | be asked to rate the severity of stinging/burning using a four-point scale |
| | (0 = none, 1 = minimal, 2 = moderate, and 3 = severe) and pruritus using a |
| | four-point scale (0 = none, 1 = mild, 2 = moderate, and 3 = severe). Only |
| | LSRs that require medical intervention (e.g., prescription medication) or |
| | require withholding the application of the test article will be documented as |
| | adverse events. Any LSRs that are not listed above will be recorded as |
| | adverse events. At each visit during the study adverse reactions, |
| | concurrent procedures and changes in concomitant medications during the |
| | study will be recorded on the source documentation. |
| | Visit 2 End of Treatment Day 29 (±3 days): |
| | The final application of the test article will be on the day immediately |
| | preceding the end of treatment visit. At this visit, clinical evaluations |
| | (erythema, scale, IGA, LSRs on the face) will be performed and a |
| | photograph documenting end of treatment will be taken of the face. |
| | Local site reactions (burning, stinging, and pruritus) will be assessed. Any |
| | adverse events and changes to concomitant medications will be noted in the |
| | study documents prior to the subject leaving the clinic. |
| | The investigator will complete an End of Study form for all completed and |
| | discontinued subjects. At the end of study visit, the test article will be |
| | dispensed to the patient to continue use until the supply is exhausted. |
| Study Measurements | Efficacy: Clinical evaluations (erythema, scale, IGA, LSRs on the face) will |
| | be performed at this visit. A grade 0 or 1 (clear or almost clear) on the IGA |
| | scale would be considered a treatment success. |
| | Safety: |
| | All adverse events (AEs) will be recorded. At each visit, subjects will also |
| | be questioned specifically about any adverse events associated with the |
| | application of the test article as well as the status of any ongoing adverse |
| | events. Frequency of LSRs during the treatment period including erythema, |
| | burning, stinging, and pruritus will also be documented. |
| Study Endpoints | Efficacy Endpoint(s): |
| | Primary efficacy endpoints will include: The change of IGA from Baseline |
| | to Day 29 will be the primary efficacy variable |
| | Secondary efficacy endpoints will include: percentage of patients that |
| | complete the study with facial clearance |
| | Safety Endpoint(s): |
| | Tolerability of treatment, local and systemic adverse events. |

¹Defined as amenorrhea greater than 12 consecutive months.
²Hysterectomy, bilateral tubal ligation (at least 6 months prior to initiation of treatment) or bilateral oophorectomy.
³Effective forms of birth control include hormonal contraceptives [oral, injectable, transdcrmal or intravaginal]³ or intrauterine device (IUD) for at least one week prior to test article application, condom and spermicidal or diaphragm and spermicidal). Other acceptable forms of birth control include: a) abstinence for subjects who are not sexually active or b) if the subject is in a monogamous relationship with a partner who is sterile (e.g., vasectomy performed at least six months prior to the subject’s initiation of treatment). Subjects who become sexually active or begin to have relations with a partner who is not sterile during the trial must agree to use an effective form of birth control for the duration of the study.
⁴Women of childbearing potential (WOCBP) taking hormonal therapy must be on treatment for at least 12 weeks prior to study entry and must not change their dosing regimen during the study. Those who have used hormonal therapy prior to study entry must have discontinued use at least eight (8) weeks prior to the start of the study.
⁵Urine pregnancy tests must have a minimum sensitiv ity of 25 mIU β-hCG/mL.

### Example 4: Clinical Study Compared to Pimecrolimus 1% Cream

A clinical trial is conducted as described in Table 3 below:

TABLE 3
Protocol Number: TI Project Number: CBD vs. Pimecrolimus Cream

| | |
|---|---|
| Title | A Single Center, Blinded, Randomized, Controlled Study to assess the |
| | comparative efficacy of the split body areas using Canno cream vs. |
| | Pimecrolimus 1% cream for atopic dermatitis. |
| Study Type | Open label proof of concept |
| Test Articles | The study includes the following two test articles: |
| | Canno cream once daily |
| | Pimecrolimus 1% cream once daily |
| Study Objective | The primary objective of this study is to compare the safety and efficacy of |
| | split surface treatment of Canno cream vs. Pimecrolimus 1% cream for the |
| | treatment of atopic dermatitis. |
| Study Design | Single-center, blinded, bilateral comparison |
| Treatment Groups | Eligible subjects will be randomly assigned to a treatment group and will |
| | receive one of the following treatments: |
| | 1. Canno cream |
| | 2. Pimecrolimus 1% cream application |
| | All subjects will apply the IP to the split symmetrical body surface areas |
| | for 4 weeks. |
| | After enrollment in the study, each subject will be assigned test article will |
| | be applied for 4 (four) weeks. |
| Duration of Treatment | Daily application for 4 weeks |
| Duration of Study | Up to eight (8) weeks |
| Study Population | Male and female subjects 18 years or older with mild to moderate acne |
| | vulgaris (Grade 2 or 3 on the Investigator’s Global Assessment) on the right |
| | and left side of the affected body surface areas. |
| Total Number of | Approximately twenty (20) subjects will be enrolled, |
| Subjects | |
| Number of Sites | One (1) US Site |
| Inclusion Criteria | To enter the study, a subject must meet the following criteria: |
| <to be revised as | 7. Subject is 18 years of age (inclusive) or older at the time of consent. |
| necessary> | 8. Subject must provide written and verbal informed consent and |
| | privacy authorization prior to participation in the study. |
| | 9. Females must be post-menopausal⁶, surgically sterile⁷ or using |
| | highly effective birth control methods⁸,⁹ with a negative urine |
| | pregnancy test (UPT) at the Baseline Visit. |
| | 10. Subject must have a diagnosis of mild to moderate atopic dermatitis |
| | (Grade 2 or 3) with involvement of both the right and left of |
| | symmetric body surface areas as determined by the Investigator’s |
| | Global Assessment (IGA). |
| | 11. Subject must have both a minimum of 2% BSA covered in |
| | designated treatment areas at the Screening Visit with similar lesion |
| | involvement present on the left and right side of the body. |
| | 12. Subject must be in general good health in the opinion of the |
| | investigator. |
| | 13. |
| Exclusion | Criteria A subject is ineligible to enter the study if he/she meets one or |
| <to be revised as | more of the following criteria: |
| necessary> | 14. Female subject who is pregnant, lactating or planning a pregnancy |
| | during the study period. |
| | 15. Subject is currently enrolled in an investigational drug or device |
| | study. |
| | 16. Subject has used topical medications dedicated to the treatment of |
| | atopic dermatitis containing steroids, calcineurin inhibitors, |
| | crisaborole ointment, antifungal agents, or any other treatments |
| | containing CBD within one week of Baseline. |
| | 17. Subject has used recreational marijuana or any other form of |
| | cannabis, cannabinoids, or similar products within 2 weeks of |
| | baseline visit. |
| | 18. Subject has any skin or medical condition, including facial hair, |
| | which could interfere with the evaluation of the study drugs. |
| | 19. Subjects who have significant neurological conditions (Parkinson’s |
| | disease or Stroke), who in the opinion of the investigator are not |
| | eligible for the study due to the severity of neurological condition |
| | to determine severity or ability to complete of trial. |
| | 20. Subject have any history of overt bacterial, viral or fungal infection |
| | of the head/neck. |
| | 21. Subject has any presence of compromising dermatosis elsewhere |
| | on the skin of the face |
| | 22. Subject has excessively actinically damaged skin in the opinion of |
| | the investigator or history of NMSC of the face within 6 months. |
| | 23. Subject plans to be exposed to artificial tanning devices or |
| | excessive sunlight during the study. |
| | 24. Light treatments, microdermabrasion or chemical peels to the face, |
| | chest and back within four (4) weeks of Baseline. |
| | 25. Subject has a history of alcohol and/or drug abuse or is unreliable |
| | in the investigator’s judgment. |
| Study Procedures | The study will consist of a Screening/Baseline Visit and X scheduled visits. |
| | 1. Visit 1 (Screening/Baseline): At Visit 1, the study staff will explain the |
| | study procedures and an informed consent must be signed prior to the |
| | initiation of any study-related procedures. At this visit, consenting subjects |
| | will have their medical history, dermatologic exam, and |
| | inclusion/exclusion (I/E) criteria reviewed to determine subject eligibility. |
| | A urine pregnancy test [UPT] (if applicable), Fitzpatrick Skin Type |
| | assessment, clinical evaluations (IGA, LSRs on the right and left side of the |
| | body treatment areas) will be performed at this visit. Prior and |
| | concomitant therapy and concomitant medications/procedures will be |
| | reviewed. Subjects who require a “washout” period for longer than 30 |
| | days prior to baseline will be re-consented. Subjects will be instructed on |
| | the proper application of the study medication. The first dose of IP will be |
| | applied in the clinic with the assistance of the clinic staff The subject will |
| | be scheduled for the first follow-up visit. |
| | 2. Visits 3, and 4 (Days 29, and 43): Subjects will return after the initial |
| | treatment for clinical evaluations of the right and left side of the body |
| | (IGA, AEs and LSRs). The IP will be applied by the subject in the clinic |
| | after clinical evaluations. |
| Study Measurements | Efficacy will be assessed on the right and left side of the body treatment |
| | areas by the investigator as follows: |
| | EAST score |
| | Investigator’s Global Assessment (IGA) |
| | Safety will be assessed by the investigator via the evaluation of LSRs and |
| | AEs at each visit. |
| Study Endpoints | Efficacy: |
| | Patients’ lesion severity, including BSA and EAST score, will be evaluated |
| | on the face by the investigator. |
| | Treatment success will be defined as |
| | Complete clearance: 100% clearance of disease activity and inflammation |
| | Partial clearance: ≥75% clearance of disease activity and inflammation |
| | Secondary measures will include subjective assessments of itching, pain |
| | and perilesional erythema at each visit. |
| | The incidence of treatment-emergent adverse events (including LSRs) |
| | will be assessed. |
| | Safety Endpoint(s): |
| | Tolerability of treatment, local and systemic adverse events. |
| Sample Size | No formal calculations of sample size were performed for this pilot study. |
| Calculations | |
| Statistical Methods | All statistical processing will be performed using SAS ® unless otherwise |
| | stated. Statistical significance for the primary efficacy endpoint will be based |
| | on two-tailed tests of the null hypothesis resulting in p-values of 0.05 or less. |
| | Summary tables (descriptive statistics and/or frequency tables) will be |
| | provided for baseline variables, efficacy variables and safety variables. |
| | Continuous variables will be described by descriptive statistics (n, mean, |
| | median, standard deviation, minimum, and maximum). Frequency counts and |
| | percentage of subjects within each category will be provided for categorical |
| | data. Summaries will be provided for each treatment group. |
| | Study Populations: |
| | All randomized subjects who received and applied <or administered> test |
| | article will be included in the analysis of safety and will be considered the |
| | Safety population. All randomized subjects who were dispensed the test article |
| | will be included in the analysis of efficacy and will be considered the intent- |
| | to-treat (ITT) population. Subjects that completed the study without significant |
| | protocol deviations will be included in the per-protocol (PP) efficacy analyses. |
| | Last-observation-carried-forward (LOCF) will be used to impute missing |
| | values for efficacy variables. |
| | Safety Analyses: |
| | The analysis of safety will be conducted on the Safety population. |
| | Dosing Compliance |
| | Descriptive statistics will be used to summarize test article compliance for |
| | the ITT population. Measures of test article compliance will include the |
| | duration of treatment, the total amount of test article used, and the total |
| | number of applications <or administrations>. |
| | Adverse Events |
| | All adverse events reported during the study will be listed, documenting |
| | course, severity, investigator assessment of the relationship to the test |
| | articles, and outcome. Verbatim terms on the case report forms (CRFs) will |
| | be linked to preferred terms (PTs) and system organ class (SOC) using the |
| | MedDRA mapping system. The PTs and SOC will then be tabulated. All |
| | reported adverse events will be summarized by the number of subjects |
| | reporting adverse events, SOC, PT, severity, and relationship to test article |
| | by treatment. |

⁶Defined as amenorrhea greater than 12 consecutive months.
⁷Hysterectomy, bilateral tubal ligation (at least 6 months prior to study entry) or bilateral oophorectomy.
⁸Effective forms of birth control include hormonal contraceptives [oral, injectable, transdermal or intravaginal]⁸ for at least 3 months prior to the Baseline Visit or intrauterine device (IUD) for at least one week prior to the Baseline Visit, condom and spermicidal or diaphragm and spermicidal). Other acceptable forms of birth control include: a) abstinence for subjects who are not sexually active or b) if the subject is in a monogamous relationship with a partner who is sterile (e.g., vasectomy performed at least six months prior to the Baseline Visit). Subjects who become sexually active or begin to have relations with a partner who is not sterile during the trial must agree to use an effective form of birth control for the duration of the study.
⁹ Women of childbearing potential (WOCBP) taking hormonal therapy must be on treatment for at least 12 weeks prior to study entry and must not change their dosing regimen during the study. Those who have used hormonal therapy prior to study entry must have discontinued use at least eight (8) weeks prior to the start of the study.

1. A lipid-based nanoparticle, comprising (i) cannabidiol and (ii) at least one fractionated oil, wherein the fractionated oil comprises at least one short-chain fatty acids having between 6 and 8 carbon items and does not contain any fatty acids having greater than 8 carbon atoms.

2. The lipid-based nanoparticle of claim 1, wherein the at least one fractionated oil is coconut oil.

3. The lipid-based nanoparticle of claim 1, which is suitable to provide for increased skin permeability and bioavailability of the cannabidiol and other cannabinoids.

4. The lipid-based nanoparticle of claim 1, further comprising at least one additional cannabinoid.

5. The lipid-based nanoparticle of claim 1, in the form of a liposome or lipid-drug conjugate.

6. The lipid-based nanoparticle of claim 1, in the form of a solid lipid nanoparticle (SLN).

7. The lipid-based nanoparticle of claim 1, in the form of a nanostructured lipid particle (NLP).

8. The lipid-based nanoparticle of claim 1, wherein the nanoparticle is about 50 nms or less.

9. The lipid-based nanoparticle formulation of claim 1, further comprising one or more penetration enhancers.

10. The liquid-based nanoparticle of claim 9, wherein the penetration enhancer is selected from the group consisting of transcutol, pyrrolidone, azone, methanol, eucalyptol, nicotinamide, glycerol, mono-, di- or polyglycols, ethyl acetate, eugenol, propylene glycol, isopryl myristate and isopropyl palmitate.

11. The lipid-based nanoparticle of claim 9, wherein the one or more penetration enhancers are present in an amount between about 0.5% and about 30%.
